SEQUENCE LISTING (10 sequences)

SEQ ID NO: 1 - protein, 26 aa, Artificial Sequence
  Synthetic Polypeptide
MKIKTGARILALSALTTMMFSASALA

SEQ ID NO: 2 - protein, 21 aa, Artificial Sequence
  Synthetic Polypeptide
MKQSTIALALLPLLFTPVTKA

SEQ ID NO: 3 - protein, 25 aa, Artificial Sequence
  Synthetic Polypeptide
MMITLRKLPLAVAVAAGVMSAQAMA

SEQ ID NO: 4 - protein, 23 aa, Artificial Sequence
  Synthetic Polypeptide
MNKKVLTLSAVMASMLFGAAAHA

SEQ ID NO: 5 - protein, 22 aa, Artificial Sequence
  Synthetic Polypeptide
MKYLLPTAAAGLLLLAAQPAMA

SEQ ID NO: 6 - protein, 19 aa, Artificial Sequence
  Synthetic Polypeptide
MKKIWLALAGLVLAFSASA

SEQ ID NO: 7 - protein, 23 aa, Artificial Sequence
  Synthetic Polypeptide
MMTKIKLLMLIIFYLIISASAHA

SEQ ID NO: 8 - protein, 21 aa, Artificial Sequence
  Synthetic Polypeptide
MKQALRVAFGFLILWASVLHA

SEQ ID NO: 9 - protein, 18 aa, Artificial Sequence
  Synthetic Polypeptide
MRVLLFLLLSLFMLPAFS

SEQ ID NO: 10 - protein, 39 aa, Artificial Sequence
  Synthetic Polypeptide
MANNDLFQASRRRFLAQLGGLTVAGMLGPSLLTPRRATA

(19) United States
(12) Patent Application Publication
(10) Pub. No.: US 2018/0273985 A1
(43) Pub. Date: Sep. 27, 2018

(54) CELL-FREE PRODUCTION OF BUTANOL

(71) Applicant: William Jeremy BLAKE, Winchester, MA (US)

(72) Inventor: William Jeremy BLAKE, Winchester, MA (US)

(21) Appl. No.: 15/559,126

(22) PCT Filed: Mar. 18, 2016
(86) PCT No.: PCT/US16/23173

§ 371 Date: Sep. 18, 2017

Related U.S. Application Data
(60) Provisional application No. 62/135,621, filed on Mar. 19, 2015.

(51) Int. Cl. C12P 7/16; C12N 1/06; C12N 9/04; C12N 9/10; C12N 9/88

(52) U.S. Cl. CPC C12P 7/16; C12N 1/066; C12N 9/0006; C12Y 101/01086; C12Y 101/01001; C12Y 202/01006; C12N 9/88; C12Y 402/01009; C12Y 401/01001; C12N 9/1022

(57) ABSTRACT

Provided herein, in some aspects, are methods and compositions for producing large-scale quantities of butanol, including normal butanol (n-butanol), isobutanol, and 2-butanol using a cell-free system.

### BACKGROUND OF INVENTION

The sustainable production of biofuels remains one of the major focuses in the field of biotechnology. Normal butanol and isobutanol are among the most desirable molecules for production. Relative to ethanol, normal butanol and isobutanol have superior liquid-fuel characteristics. For example, normal butanol has similar properties to gasoline; thus, it has the potential to be used as a substitute for gasoline. Metabolic engineering permits production of biofuels, such as butanol, through manipulation of biosynthetic pathways in a cell. Despite advances in metabolic engineering, large-scale production of biofuels remains challenging.

### BRIEF SUMMARY OF INVENTION

Biofuels, such as “biobutanor”—a term used to describe butanol (e.g., normal butanol, isobutanol, and 2-butanol) produced from biomass feedstocks—offer an alternative to conventional transportation fuel. The benefits of biobutanol, for example, include higher energy content, increased energy security, and fewer emissions. Nonetheless, large-scale production of biofuels remains challenging, in part because most linear and aromatic hydrocarbons generated during the production of biofuels are strong inhibitors of cell growth. For example, concentrations of isobutanol as low as 1-2% (v/v) can induce toxic effects in a microbial production host, reducing both cellular growth rates and isobutanol precursor synthesis, resulting in low product yields.

Provided herein, in some aspects, are methods, systems, compositions, and kits (e.g., cell lysates) for producing large-scale quantities and/or high titers (e.g., greater than 5% v/v) of butanol, including normal butanol (n-butanol), isobutanol, and 2-butanol, using a cell-free process, whereby butanol is synthesized by enzymes of a butanol biosynthetic pathway in a cell-free reaction containing, for example, glucose or pyruvate. Cells are first typically cultured to produce the enzymes in the butanol biosynthetic pathway, and then the cells are lysed for the butanol-production phase.

Thus, some aspects of the present disclosure are directed to methods of producing a cell lysate for producing n-butanol, isobutanol, or 2-butanol.

In some embodiments, the methods comprise culturing engineered cells that express enzymes of a butanol biosynthetic pathway under conditions that result in expression of those enzymes, and then lysing the cultured engineered cells to produce a cell lysate that comprises the enzymes of the butanol biosynthetic pathway.

In some embodiments, the butanol biosynthetic pathway is a normal butanol (n-butanol) biosynthetic pathway, and the enzymes of the n-butanol biosynthetic pathway are glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase (see Table 1 and Table 2).

In some embodiments, the butanol biosynthetic pathway is an isobutanol biosynthetic pathway, and the enzymes of the isobutanol biosynthetic pathway are glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase (e.g., Bacillus subtilis acetolactate synthase), ketol-acid reductoisomerase (e.g., mutated NADH-dependent Escherichia coli ketol-acid reductoisomerase), dihydroxy-acid dehydratase (e.g., Streptococcus nutans dihydroxy-acid dehydratase), branched-chain-2-oxoacid decarboxylase (e.g., Lactococcus lactis α-ketoisovalerate decarboxylase), and alcohol dehydrogenase (e.g., Achromobacter xylosoxidans butanol dehydrogenase) (see Table 1 and Table 3).

In some embodiments, the butanol biosynthetic pathway is a 2-butanol biosynthetic pathway, and the enzymes of the 2-butanol biosynthetic pathway are glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, acetolactate decarboxylase, diacetyl reductase, diol dehydratase or glycerol dehydratase, and butanol dehydrogenase (see Table 1 and Table 4).

In some embodiments, cell lysates comprising enzymes of a butanol biosynthetic pathway are combined with glucose and incubated under conditions that result in the production of butanol (e.g., n-butanol, isobutanol, or 2-butanol).

Other aspects of the present disclosure are directed to methods of producing a cell lysate for producing an intermediate in a butanol biosynthetic pathway, such as pyruvate. Thus, in some embodiments, methods as provided herein comprise culturing engineered cells that express enzymes of the glycolytic pathway, and lysing the cultured engineered cells to produce a cell lysate that comprises enzymes of the glycolytic pathway. In some embodiments, the enzymes of the glycolytic pathway are glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase.

In some embodiments, cell lysates comprising enzymes of the glycolytic pathway are combined with glucose and incubated under conditions that result in the production of pyruvate. The pyruvate can then be used to produce butanol or other compounds. Thus, in some embodiments, provided herein are methods of producing a cell lysate for producing pyruvate, the method comprising: (a) culturing engineered cells that express at least one (e.g., at least 2, 3, 4, 5, 6, 7, 8, 9, or 10) enzyme of a pyruvate pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase, wherein the cells are cultured under conditions that result in expression of enzymes, and (b) lysing engineered cells cultured in step (a), thereby producing a cell lysate that comprises at least one enzyme of the pyruvate pathway. In some embodiments, the cell lysate is incubated under conditions that result in production of pyruvate. In some embodiments, at least one cell lysate comprising pyruvate is combined with at least one cell lysate comprising at least one enzyme of a n-butanol, isobutanol, or 2-butanol pathway. In other embodiments, pyruvate is isolated from the cell lysate and combined with at least one cell lysate comprising at least one enzyme of a n-butanol, isobutanol, or 2-butanol pathway.

The details of several embodiments of the invention are set forth in the accompanying Figures and the Detailed Description. Other features, objects, and advantages of the invention will be apparent from the description and from the claims.

### BRIEF DESCRIPTION OF DRAWINGS

FIG. 1A shows the chemical structure of n-butanol.

FIG. 1B shows a schematic for the biosynthetic production of n-butanol using glucose as the starting substrate.

FIG. 2A shows the chemical structure of isobutanol.

FIG. 2B shows a schematic for the biosynthetic production of isobutanol using glucose as the starting substrate.

FIG. 3A shows the chemical structure of 2-butanol.

FIG. 3B shows a schematic for the biosynthetic production of 2-butanol using glucose as the starting substrate.

FIG. 4 shows a pSP62 plasmid containing five of the genes encoding the five enzymes used to catalyze conversion of pyruvate into isobutanol. The block arrows indicate open reading frames (ORFs). The small black arrows indicate T7 promoters fused with lac operator sites. The octagons indicate T7 terminators. The block indicates origin of replication (ori).

FIG. 5 shows a graph of isobutanol production over time for the BL21 DE3 pSP62 cell-free extract pyruvate→isobutanol assay. One replicate was conducted for time 0. Two replicates were conducted for time points at 1, 2, and 3 minutes. The error bars represent +/−standard deviation.

FIG. 6 shows a graph of isobutanol production over time for the BL21 DE3 pSP62 cell-free extract glucose→isobutanol assay. One replicate was conducted for time 0. Two replicates were conducted for time points at 6, 12, and 18 minutes. The error bars represent +/−standard deviation.

### DETAILED DESCRIPTION OF INVENTION

Butanol produced biologically (e.g., from biomass, such as lignocellulosic biomass, or derivatives thereof, such as glucose) is a renewable alternative to petroleum-based chemical commodities and fuels. Nonetheless, fermentation-based production of butanol is limited by the low tolerance of microbial production systems to the end products. Provided herein are cost-effective, efficient methods for the large-scale production of butanol from glucose using a cell-free system.

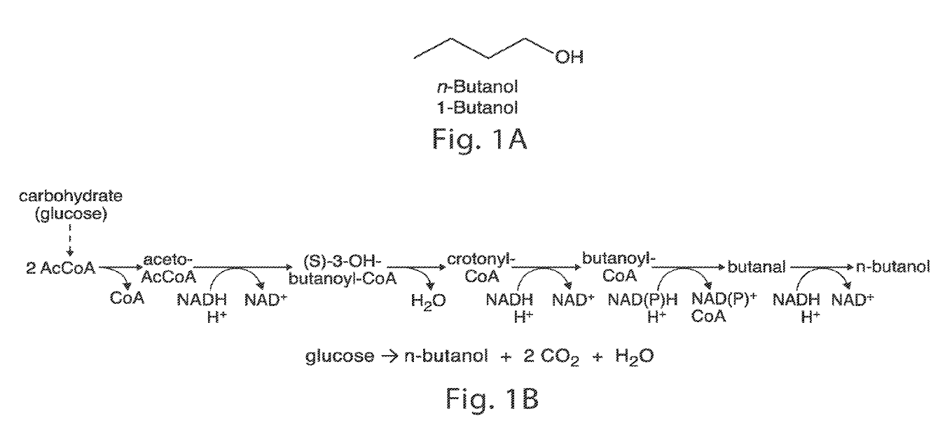

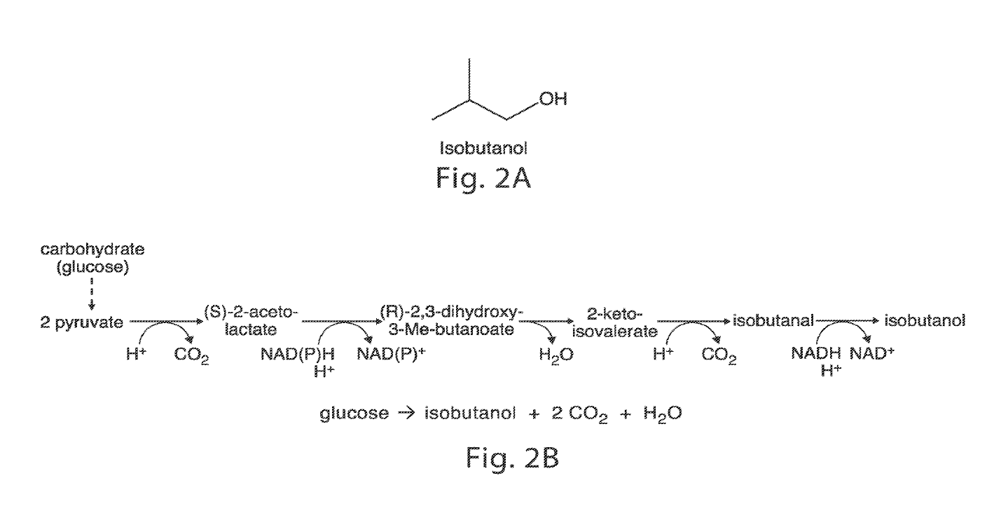

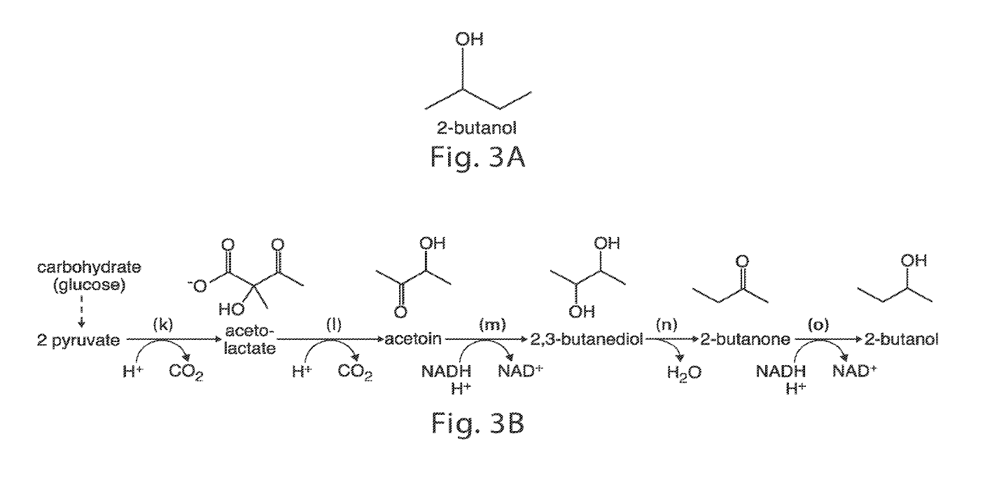

“Butanol,” as used herein, refers to a four-carbon alcohol with a formula of C₄H₉OH and includes isomeric structures, such as normal butanol and isobutanol. Normal butanol (n-butanol), also referred to as 1-butanol, is a straight chain isomer with an alcohol functional group at a terminal carbon (FIG. 1A). Isobutanol, also referred to as 2-methyl-1-propanol, is a branched isomer with an alcohol at a terminal carbon (FIG. 2A). 2-butanol, also referred to as sec-butanol, is chiral and, thus, can be obtained as either of two stereoisomers designated as (R)-(−)-2-butanol and (S)-(+)-2-butanol (FIG. 3A).

In some aspects of the present disclosure, methods of producing butanol include expressing in engineered cells enzymes of a butanol biosynthetic pathway, lysing the cells to produce cell lysates containing enzymes of the pathway, combining the cell lysates into a single reaction mixture with glucose (with or without purified enzymes of the butanol biosynthetic pathway), and incubating the reaction mixture under conditions that result in the production of butanol.

A biosynthetic pathway is a description of the steps of the chemical reactions that occur when a new molecule is created in a cell, or using cellular components (e.g., enzymes), out of precursor molecules. Biosynthetic pathways often include enzymes, co-enzymes, co-factors and substrates, for example. A “butanol biosynthetic pathway” refers to a series of chemical reactions required to synthesize butanol (e.g., n-butanol or isobutanol) from a specified substrate, such as glucose or pyruvate. “Enzymes of a butanol biosynthetic pathway” are the enzymes necessary to catalyze each chemical reaction in the pathway. Some methods of the present disclosure utilize enzymes of an n-butanol biosynthetic pathway, while other methods of the present disclosure utilize enzymes of an isobutanol biosynthetic pathway.

Examples of enzymes of an n-butanol biosynthetic pathway include glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase (see Table 1 and Table 2).

Examples of enzymes of an isobutanol biosynthetic pathway include glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, ketol-acid reductoisomerase, dihydroxy-acid dehydratase, branched-chain-2-oxoacid decarboxylase, and alcohol dehydrogenase (see Table 1 and Table 3).

Examples of enzymes of a 2-butanol biosynthetic pathway include glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, acetolactate decarboxylase, diacetyl reductase, diol dehydratase or glycerol dehydratase, and butanol dehydrogenase (see Table 1 and Table 4).

Enzymes, or nucleic acids encoding enzymes, of a butanol biosynthetic pathway (or a glycolytic pathway) may be obtained from, or endogenous to, a single organism (e.g., Escherichia or Clostridium) or multiple organisms (e.g., at least one obtained from Escherichia and at least one obtained from Clostridium).

In some embodiments, at least one (e.g., at least 2, at least 3, or at least 4) of glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase is obtained from, or endogenous to, Escherichia (e.g., E. coli). In some embodiments, at least one (e.g., at least 2, at least 3, or at least 4) of glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase is obtained from, or endogenous to, Clostridium (e.g., C. acetobutylicum, C. beijerinckii, or C. saccaroperbutylacetonicum).

In some embodiments, at least one (e.g., at least 2, at least 3, or at least 4) of pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase is obtained from, or endogenous to, Escherichia (e.g., E. coli). In some embodiments, at least one (e.g., at least 2, at least 3, or at least 4) of pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase is obtained from, or endogenous to, Clostridium (e.g., C. acetobutylicum, C. beijerinckii, or C. saccaroperbutylacetonicum).

In some embodiments, at least one (e.g., at least 2, at least 3, or at least 4) of acetolactate synthase, ketol-acid reductoisomerase, dihydroxy-acid dehydratase, branched-chain-2-oxoacid decarboxylase, and alcohol dehydrogenase is obtained from, or endogenous to, Escherichia (e.g., E. coli). In some embodiments, at least one (e.g., at least 2, at least 3, or at least 4) of acetolactate synthase, ketol-acid reductoisomerase, dihydroxy-acid dehydratase, branched-chain-2-oxoacid decarboxylase, and alcohol dehydrogenase is obtained from, or endogenous to, Clostridium (e.g., C. acetobutylicum, C. beijerinckii, or C. saccaroperbutylacetonicum).

In some embodiments, at least one (e.g., at least 2, at least 3, or at least 4) of acetolactate synthase, acetolactate decarboxylase, diacetyl reductase, diol dehydratase or glycerol dehydratase, and butanol dehydrogenase is obtained from, or endogenous to, Escherichia (e.g., E. coli). In some embodiments, at least one (e.g., at least 2, at least 3, or at least 4) of acetolactate synthase, acetolactate decarboxylase, diacetyl reductase, diol dehydratase or glycerol dehydratase, and butanol dehydrogenase is obtained from, or endogenous to, Clostridium (e.g., C. acetobutylicum, C. beijerinckii, or C. saccaroperbutylacetonicum).

Enzymes of butanol biosynthetic pathways are discussed in more detail below.

It should be understood that variants of enzymes of a butanol biosynthetic pathway may be used as provided herein. A variant enzyme may be, for example, at least 80% homologous to an enzyme of a butanol (e.g., n-butanol, isobutanol, or isobutanol, or 2-butanol) biosynthetic pathway. In some embodiments, a variant enzyme is 80%, 85%, 90%, 95%, 96%, 97%, 98%, or 99% homologous to an enzyme of a butanol biosynthetic pathway. A variant of an enzyme may be used in accordance with the present disclosure provided that the variant has the same, or similar, activity as the enzyme.

Butanol, as provided herein, can be produced using the inventive system from glucose or pyruvate, or other intermediates in the pathway from glucose to butanol. Glucose (C₆H₁₂O₆), also referred to as dextrose, is converted to pyruvate via glycolysis. The enzymes, substrates and products involved in glycolysis are listed in Table 1. It should be understood that each of the butanol biosynthetic pathways provided herein utilize enzymes of the glycolytic pathway for converting glucose into pyruvate.

### Glycolysis

Butanol, including n-butanol, isobutanol, and 2-butanol, in some embodiments, is produced biosynthetically from glucose through the glycolytic pathway (i.e., glycolysis)—the metabolic pathway that converts glucose to pyruvate.

Enzymes of the glycolytic pathway include glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase (Table 1). Thus, aspects of the present disclosure comprise culturing engineered cells that express enzymes of glycolytic pathway selected from the group consisting of glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase. Engineered cells of the present disclosure may endogenously or exogenously express one or more, or all, of the enzymes of the glycolytic pathway. For example, engineered cells may express 1, 2, 3, 4, 5, 6, 7, 8, 9, or 10 enzymes of the glycolytic pathway.

Enzymes of the glycolytic pathway may be obtained from, or endogenous to, a single organism or multiple organisms. For example, engineered cells (e.g., Escherichia cells) of the present disclosure may endogenously express enzymes of the glycolytic pathway, and/or at least some of the enzymes (e.g., engineered enzymes) of the glycolysis pathway may be obtained from one or more different organisms (e.g., Clostridium).

TABLE 1
Enzymes Used for the Conversion of Glucose to Pyruvate

| | EC | | | |
|---|---|---|---|---|
| Step | Number | Activity | Substrate | Product |
| a | 2.7.1.2 | glucokinase | Glucose | glucose-6-phosphate |
| b | 5.3.1.9 | phosphoglucose | glucose-6-phosphate | fructose-6-phosphate |
| | | isomerase | | |
| c | 2.7.1.11 | phosphofructokinase | fructose-6-phosphate | fructose 1,6- |
| | | | | bisphosphate |
| d | 4.1.2.13 | fructose bisphosphate | fructose 1,6- | dihydroxyacetone |
| | | aldolase | bisphosphate | phosphate, |
| | | | | glyceraldehyde 3- |
| | | | | phosphate |
| e | 5.3.1.1 | triose phosphate | dihydroxyacetone | glyceraldehyde 3- |
| | | isomerase | phosphate | phosphate |
| f | 1.2.1.12 | glyceraldehyde 3- | glyceraldehyde 3- | 1,3-bisphospho-D- |
| | | phosphate dehydrogenase | phosphate | glycerate |
| | | (GAPDH) | | |
| g | 2.7.2.3 | phosphoglycerate kinase | 1,3-bisphosphoglycerate | 3-phospho-D-glycerate |
| h | 5.4.2.1 | phosphoglycerate mutase | 3-phospho-D-glycerate | 2-phospho-D-glycerate |
| i | 4.2.1.11 | enolase | 2-phospho-D-glycerate | phosphoenolpyruvate |
| j | 2.7.1.40 | pyruvate kinase | phosphoenolpyruvate | pyruvate |

Glucokinase is an enzyme (EC 2.7.1.2) that facilitates phosphorylation of glucose to glucose-6-phosphate and is active in the following biological pathways: glycolysis/gluconeogenesis, galactose metabolism, starch and sucrose metabolism, amino sugar and nucleotide sugar metabolism, streptomycin biosynthesis, butirosin and neomycin biosynthesis, metabolic pathways, biosynthesis of secondary metabolites, and microbial metabolism. Glucokinase belongs to the following classes of enzymes: transferases, transferring phosphorus-containing groups, and phosphotransferases having an alcohol group as an acceptor. See Baumann, Biochemistry 8 (1969) 5011-5, Bueding et al., J. Biol. Chem. 215 (1955) 495-506, and Porter et al., Biochim. Biophys. Acta. 709 (91982) 178-86, each of which is incorporated by reference herein.

Glucose-6-phosphate isomerase is an enzyme (EC 5.3.1.9) that catalyzes the conversion of glucose-6-phosphate into fructose 6-phosphate (as well as the anomerization of D-glucose 6-phosphate) and is active in the following biological pathways: glycolysis/gluconeogenesis, the pentose phosphate pathway, starch and sucrose metabolism, amino sugar and nucleotide sugar metabolism, metabolic pathways, biosynthesis of secondary metabolites, and microbial metabolism. Glucose-6-phosphate isomerase is also referred to as phosphoglucose isomerase, phosphohexomutase, oxoisomerase, hexosephosphate isomerase, phosphosaccharomutase, phosphoglucoisomerase, phosphohexoisomerase, phosphoglucose isomerase, glucose phosphate isomerase, hexose phosphate isomerase, or D-glucose-6-phosphate ketol-isomerase. Glucose-6-phosphate isomerase belongs to the following classes of enzymes: isomerases, intramolecular oxidoreductases, interconverting aldoses and ketoses, and related compounds. See Baich et al., J. Biol. Chem. 235 (1960) 3130-3, Nakagawa et al., J. Biol. Chem. 240 (1965) 1877-81, Noltmann, Biochem. Z. 331 (1959) 436-445, Ramasarma et al., Arch. Biochem. Biophys. 62 (1956) 91-6, and Tsuboi et al., J. Biol. Chem. 231 (1958) 19-29, each of which is incorporated by reference herein.

Phosphofructokinase is a kinase enzyme (EC 2.7.1.11) that phosphorylates fructose 6-phosphate to produce fructose 1,6-bisphosphate and is active in the following biological pathways: glycolysis/gluconeogenesis, the pentose phosphate pathway, fructose and mannose metabolism, galactose metabolism, methane metabolism, metabolic pathways, biosynthesis of secondary metabolites, and microbial metabolism. Phosphofructokinase is also referred to as 6-phosphofructokinase, phosphohexokinase, phosphofructokinase I, phosphofructokinase (phosphorylating), 6-phosphofructose 1-kinase, ATP-dependent phosphofructokinase, D-fructose-6-phosphate 1-phosphotransferase, fructose 6-phosphate kinase, fructose 6-phosphokinase, nucleotide triphosphate-dependent phosphofructokinase, phospho-1,6-fructokinase, or PFK. See Axelrod et al., J. Biol. Chem. 197 (1952) 89-96, Ling et al., Methods Enzymol. 9 (1966) 425-429, Mansour, Methods Enzymol. 9 (1966) 430-436, Odeide et al., Bull. Soc. Chim. Biol. (Paris). 50 (1968) 2023-33, Parmeggiani et al., J. Biol. Chem. 241 (1966) 4625-37, Racker, J. Biol. Chem. 167 (1947) 843-854, Sols et al., Methods Enzymol. 9 (1966) 436-442, and Uyeda et al., J. Biol. Chem. 245 (1970) 3315-24, each of which is incorporated by reference herein.

Fructose-bisphosphate aldolase is an enzyme (EC 4.1.2.13) that catalyzes a reversible reaction that splits the aldol in fructose 1,6-bisphosphate, into the triose phosphates dihydroxyacetone phosphate (DHAP) and glyceraldehyde 3-phosphate (GAP). Fructose-bisphosphate aldolase also acts on (3S,4R)-ketose 1-phosphates. The enzyme increases carbonyl group electron attraction by either forming a protonated imine with it (Class I), or polarizing it with a metal ion, such as zinc (Class II, generally of microbial origin). Fructose-bisphosphate aldolase is active in the following biological pathways: glycolysis/gluconeogenesis, pentose phosphate pathway, fructose and mannose metabolism, methane metabolism, carbon fixation in photosynthetic organisms, metabolic pathways, biosynthesis of secondary metabolites, and microbial metabolism. Fructose-bisphosphate aldolase is also referred to as aldolase, fructose-1,6-bisphosphate triosephosphate-lyase, fructose diphosphate aldolase, diphosphofructose aldolase, fructose 1,6-diphosphate aldolase, ketose 1-phosphate aldolase, phosphofructoaldolase, zymohexase, fructoaldolase, fructose 1-phosphate aldolase, fructose 1-monophosphate aldolase, 1,6-diphosphofructose aldolase, SMALDO, or D-fructose-1,6-bisphosphate D-glyceraldehyde-3-phosphate-lyase. Fructose-bisphosphate aldolase is included in the following classes of enzymes: lyases, carbon-carbon lyases, and aldehyde-lyases. See Horecker et al., in: Boyer, P. D. (Ed.), The Enzymes, 3rd ed., vol. 7, Academic Press, New York, 1972, p. 213-258, and Alefounder et al., Biochem. J. 257 (1989) 529-34, each of which is incorporated by reference herein.

Triose-phosphate isomerase (TPI or TIM) is an enzyme (EC 5.3.1.1) that catalyzes the reversible interconversion of the triose phosphate isomers dihydroxyacetone phosphate and D-glyceraldehyde 3-phosphate and is active in the following biological pathways: glycolysis/gluconeogenesis, fructose and mannose metabolism, inositol phosphate metabolism, carbon fixation in photosynthetic organisms, metabolic pathways, biosynthesis of secondary metabolites, and microbial metabolism. Triose-phosphate isomerase is also referred to as phosphotriose isomerase, triose phosphoisomerase, triose phosphate mutase, or D-glyceraldehyde-3-phosphate ketol-isomerase, and belongs to the following classes: isomerases, intramolecular oxidoreductases, and interconverting aldoses and ketoses, and related compounds. See Meyer-Arendt et al., Naturwissenschaften 40 (1953) 59 and Meyerhof et al., J. Biol. Chem. 156 (1944) 109-120, each of which is incorporated by reference herein.

Glyceraldehyde 3-phosphate dehydrogenase (GAPDH) is an enzyme (EC 1.2.1.12) that catalyzes the conversion of glyceraldehyde 3-phosphate to 1,3-bisphosphoglycerate (1,3-BPG), and is active in the following biological pathways: glycolysis/gluconeogenesis, carbon fixation in photosynthetic organisms, metabolic pathways, biosynthesis of secondary metabolites, and microbial metabolism. GAPDH is also referred to as triosephosphate dehydrogenase, dehydrogenase, glyceraldehyde phosphate, phosphoglyceraldehyde dehydrogenase, 3-phosphoglyceraldehyde dehydrogenase, NAD⁺-dependent glyceraldehyde phosphate dehydrogenase, glyceraldehyde phosphate dehydrogenase (NAD⁺), glyceraldehyde-3-phosphate dehydrogenase (NAD⁺), NADH-glyceraldehyde phosphate dehydrogenase, or glyceraldehyde-3-P-dehydrogenase. GAPDH belongs to the following classes: oxidoreductases, acting on the aldehyde or oxo group of donors, and with NAD⁺ and NAD⁺ as an acceptor. See Caputto et al., Nature (Lond.) 156 (1945) 630-631, Cori et al., J. Biol. Chem. 173 (1948) 605-18, Hageman et al., Arch. Biochem. 55 (1955) 162-8, Velick et al., in: Boyer, P. D., Lardy, H. and Myrback, K. (Eds.), The Enzymes, 2nd ed., vol. 7, Academic Press, New York, 1963, p. 243-273, and Warburg et al., Biochem. Z. 303 (1939) 40-68, each of which is incorporated by reference herein.

Phosphoglycerate kinase (PGK) is an enzyme (EC 2.7.2.3) that catalyzes the reversible transfer of a phosphate group from 1,3-bisphosphoglycerate (1,3-BPG) to ADP producing 3-phosphoglycerate (3-PG) and adenosine triphosphate (ATP), and is active in the following biological pathways: glycolysis/gluconeogenesis, carbon fixation in photosynthetic organisms, metabolic pathways, biosynthesis of secondary metabolites, and microbial metabolism in diverse environments. PGK is also known as 3-PGK, ATP-3-phospho-D-glycerate-1-phosphotransferase, ATP:D-3-phosphoglycerate 1-phosphotransferase, 3-phosphoglycerate kinase, 3-phosphoglycerate phosphokinase, 3-phosphoglyceric acid kinase, 3-phosphoglyceric acid phosphokinase, 3-phosphoglyceric kinase, glycerate 3-phosphate kinase, glycerophosphate kinase, phosphoglyceric acid kinase, phosphoglyceric kinase, or phosphoglycerokinase. PGK belongs to the following classes: transferases, transferring phosphorus-containing groups, and phosphotransferases with a carboxy group as an acceptor. See Axelrod et al., J. Biol. Chem. 204 (1953) 939-48, Bucher, Biochim. Biophys. Acta 1 (1947) 292-314, Hashimoto et al., Biochim. Biophys. Acta. 65 (1962) 355-7, and Rao et al., Biochem. J. 81 (1961) 405-11, each of which is incorporated by reference herein.

Phosphoglycerate mutase (2,3-diphosphoglycerate-independent) is an enzyme (EC 5.4.2.12) that catalyzes the conversion of 2-phospho-D-glycerate (2PG) to 3-phospho-D-glycerate (2PG) through a 2,3-bisphosphoglycerate intermediate, and is active in the following biological pathways: glycolysis/gluconeogenesis, glycine, serine and threonine metabolism, methane metabolism, metabolic pathways, biosynthesis of secondary metabolites, and microbial metabolism. Phosphoglycerate mutase is also known as cofactor independent phosphoglycerate mutase, 2,3-diphosphoglycerate-independent phosphoglycerate mutase, iPGM, iPGAM, or PGAM-I, and belongs in the following classes: isomerases, intramolecular transferases, and phosphotransferases (phosphomutases). See Jedrzejas, et al., J. Biol. Chem. 275 (2000) 23146-53, Rigden et al., J. Mol. Biol. 328 (2003) 909-20, Zhang et al., J. Biol. Chem. 279 (2004) 37185-90, Nukui et al., Biophys. J. 92 (2007) 977-88, Nowicki et al., J. Mol. Biol. 394 (2009) 535-43, and Mercaldi et al., FEBS. J. 279 (2012) 2012-21.

Enolase is a metalloenzyme (EC 4.2.1.11) that catalyzes the conversion of 2-phosphoglycerate (2-PG) to phosphoenolpyruvate (PEP), acts on 3-phospho-D-erythronate, and is active in the following biological pathways: glycolysis/gluconeogenesis, methane metabolism, metabolic pathways, biosynthesis of secondary metabolites, and microbial metabolism. Enolase is also referred to as phosphopyruvate hydratase, 2-phosphoglycerate dehydratase, 14-3-2-protein, nervous-system specific enolase, phosphoenolpyruvate hydratase, 2-phosphoglycerate dehydratase, 2-phosphoglyceric dehydratase, 2-phosphoglycerate enolase, gamma-enolase, or 2-phospho-D-glycerate hydro-lyase. Enolase is included in the following classes: lyases, carbon-oxygen lyases, and hydro-lyases. See Holt et al., J. Biol. Chem. 236 (1961) 3227-31, Malmstrom, in: Boyer, P. D., Lardy, H. and Myrback, K. (Eds.), The Enzymes, 2nd ed., vol. 5, Academic Press, New York, 1961, p. 471-494, and Westhead et al., J. Biol. Chem. 239 (1964) 2464-8.

Pyruvate kinase is an enzyme (EC 2.7.1.40), also known as phosphoenolpyruvate kinase or phosphoenol transphosphorylase, that catalyzes the transfer of a phosphate group from phosphoenolpyruvate (PEP) to adenosine diphosphate (ADP), yielding one molecule of pyruvate and one molecule of ATP. It is active in the following biological pathways: glycolysis/gluconeogenesis, purine metabolism, pyruvate metabolism, metabolic pathways, biosynthesis of secondary metabolites, and microbial metabolism. Pyruvate kinase belongs to the following classes: transferases, transferring phosphorus-containing groups, and phosphotransferases with an alcohol group as an acceptor. See Boyer, in: Boyer, P. D., Lardy, H. and Myrback, K. (Eds.), The Enzymes, 2nd ed., vol. 6, Academic Press, New York, 1962, p. 95-113. Kornberg et al., J. Biol. Chem. 193 (1951) 481-95, Kubowitz et al., Biochem. Z. 317 (1944) 193-203, Strominger, Biochim. Biophys. Acta. 16 (1955) 616-8, and Tietz et al., Arch. Biochem. Biophys. 78 (1958) 477-93, each of which is incorporated by reference herein.

Stoichiometry for production of two moles of pyruvate from one mole of glucose via steps (a)-(j) of Table 1 is shown below.

glucose+2ADP+2Pᵢ+2NAD⁺→2pyruvate+2ATP+2NADH+2H⁺+2H₂O

In certain embodiments, to provide a driving force for sustained pyruvate production, the nicotinamide adenine dinucleotide redox cofactor (NADH) is converted to its oxidized form (NAD⁺), and the adenosine triphosphate (ATP) is hydrolyzed to recycle adenosine diphosphate (ADP) and inorganic phosphate (Pᵢ). In instances where pyruvate is further converted through additional enzymatic steps to a final product, such as butanol, the required redox and energy cofactor turnover may be accomplished via these additional downstream enzymatic steps (e.g., if the steps require ATP or NADH). For example, n-butanol production requires (1) NADH for the conversion of acetoacetyl-CoA to 3-hydroxybutanoyl-CoA by hydroxybutyrl-CoA dehydrogenase, (2) NADH for the conversion of crotonyl-CoA to butanoyl-CoA by crotonyl-CoA reductase, NAD(P)H for the conversion of butanoyl-CoA to butyraldehyde by butyraldehyde dehydrogenase, and NADH for the conversion of butyraldehyde to n-butanol by alcohol dehydrogenase. Isobutanol production requires (1) NAD(P)H for the conversion of (S)-2-acetolactate to (R)-2,3-dihydroxy-3-methylbutanoate by ketol-acid reductoisomerase, and (2) NADH for the conversion of isobutyraldehyde to isobutanol by alcohol dehydrogenase. If the final product is pyruvate itself, the redox and energy cofactor turnover may occur through a different route.

In some embodiments of cell-free systems, NADH oxidation can be accomplished via electron transport chain complexes within inverted membrane vesicles (IMVs) (Jewett et al., Mol Syst Biol. 2008, 4:220, incorporated by reference herein). Inverted membrane vesicles form upon cell lysis and contain the required components for NADH oxidation—specifically, an NADH:ubiquinone oxidoreductase and a cytochrome terminal oxidase oxidize NADH, where molecular oxygen serves as the terminal electron acceptor and other lipid-membrane-soluble redox carriers (typically quinones) also participate. The electron transport chain generates a proton gradient across the membrane, which must dissipate to allow for continuous NADH turnover. This transmembrane proton gradient can be forced to dissipate through the addition of a proton leakage agent (e.g., dinitrophenol, carbonyl cyanide m-chlorophenyl hydrazine, carbonyl cyanide-p-trifluoromethoxyphenylhydrazone, monensin A, nigericin, or gramicidin) or enzymatically by, for example, ATP synthase that is also present in inverted membrane vesicle membranes. Thus, in some embodiments, a proton leakage agent may be added to a cell lysate or reaction mixture of the present disclosure. ATP synthase ‘pumps’ protons across the cell membrane in the opposite direction, while generating ATP from ADP and inorganic phosphate. The ATP generated by such oxidative phosphorylation, which enables NADH cofactor turnover, can be hydrolyzed along with the substrate-level ATP generated by phosphoglycerate kinase and pyruvate kinase in order to replenish ADP for sustained pyruvate production. Phosphatases that directly hydrolyze ATP or those that create energy draining phosphatase/kinase cycles, with activities present in cell extract, can be used for this purpose. Thus, in some embodiments, a phosphatase may be added to a cell lysate or reaction mixture of the present disclosure, or may be expressed from the engineered cell with or without a periplasmic leader sequence.

### Normal Butanol Biosynthetic Pathway

Some aspects of the present disclosure relate specifically to the production of normal butanol (n-butanol) from glucose, pyruvate, or other intermediates in the pathway.

Enzymes of the n-butanol biosynthetic pathway include enzymes of the glycolytic pathway (e.g., glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase; Table 1) as well as enzymes that convert pyruvate to n-butanol (e.g., pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase (Table 2)). Thus, aspects of the present disclosure comprise culturing engineered cells that express at least one (e.g., 1, 2, 3, 4, 5, 6, 7, 8, 9, 10, 11, 12, 13, 14, 15, 16, or 17) enzyme of the n-butanol biosynthetic pathway selected from the group consisting of glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase. In some embodiments, methods comprise culturing engineered cells that express at least one (e.g., 1, 2, 3, 4, 5, 6, 7, 8, 9, or 10) of glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase. In some embodiments, methods comprise culturing engineered cells that express at least one (e.g., 1, 2, 3, 4, 5, 6, 7, or 8) of pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase.

TABLE 2
Enzymes Used for the Conversion of Pyruvate to Normal Butanol

| EC | | | |
|---|---|---|---|
| Number | Activity | Substrate | Product |
| 1.2.1.— | pyruvate dehydrogenase | pyruvate | acetyl-CoA |
| | complex | | |
| 2.3.1.9 | acetyl-CoA | acetyl-CoA | acetoacetyl-CoA |
| | acetyltransferase | | |
| 1.1.1.35 | hydroxybutyrl-CoA | acetoacetyl-CoA | 3-hydroxybutanoyl-CoA |
| | dehydrogenase | | |
| 4.2.1.150 | enoyl-CoA hydratase | 3-hydroxybutanoyl-CoA | crotonyl-CoA |
| 1.3.1.44 | crotonyl-CoA reductase | crotonyl-CoA | butanoyl-CoA |
| 1.2.1.57 | butyraldehyde | butanoyl-CoA | butyraldehyde |
| | dehydrogenase | | |
| 1.1.1.1 | alcohol dehydrogenase | butyraldehyde | n-butanol |

Pyruvate dehydrogenase complex (PDC) is a complex of three enzymes (pyruvate dehydrogenase (EC 1.2.4.1), dihydrolipoyl transacetylase (EC 2.3.1.12), and dihydrolipoyl dehydrogenase (EC 1.8.1.4)) that convert pyruvate into acetyl-CoA by a process referred to as pyruvate decarboxylation.

Acetyl-CoA C-acetyltransferase (also referred to as thiolase) is an enzyme (EC 2.3.1.9) that catalyzes the conversion of acetyl-CoA to acetoacetyl-CoA and is active in the following biological pathways: fatty acid degradation, synthesis and degradation of ketone bodies, valine, leucine and isoleucine degradation, lysine degradation, benzoate degradation, tryptophan metabolism, pyruvate metabolism, glyoxylate and dicarboxylate metabolism, propanoate metabolism, butanoate metabolism, carbon fixation pathways in prokaryotes, terpenoid backbone biosynthesis, metabolic pathways, biosynthesis of secondary metabolites, and microbial metabolism. Acetyl-CoA C-acetyltransferase is also referred to as acetoacetyl-CoA thiolase, beta-acetoacetyl coenzyme A thiolase, 2-methylacetoacetyl-CoA thiolase [misleading], 3-oxothiolase, acetyl coenzyme A thiolase, acetyl-CoA acetyltransferase, acetyl-CoA:N-acetyltransferase, or thiolase II. Acetyl-CoA C-acetyltransferase belongs to the following classes: transferases, acyltransferases, and transferring groups other than aminoacyl groups. See Lynen et al., Biochim. Biophys. Acta. 12 (1953) 299-314, and Stern, Jr. et al., J. Biol. Chem. 235 (1960) 313-7. In some embodiment, the acetyl-CoA-acetyltransferase is a thermo- and solvent stable acetyl-CoA-acetyltransferase obtained from the thermophilic bacterium Meiothermus ruber (Reiße S., et al., Biochimie. 2014 August; 103:16-22, incorporated by reference herein).

Hydroxybutyryl-CoA dehydrogenase is an enzyme (EC 1.1.1.35) that catalyzes the conversion of acetoacetyl-CoA to 3-hydroxybutanoyl-CoA, oxidizes S-3-hydroxyacyl-N-acylthioethanolamine and S-3-hydroxyacyl-hydrolipoate, and is active in the following biological pathways: fatty acid elongation, fatty acid degradation, primary bile acid biosynthesis, valine, leucine and isoleucine degradation, geraniol degradation, lysine degradation, tryptophan metabolism, toluene degradation, butanoate metabolism, carbon fixation pathways in prokaryotes, caprolactam degradation, metabolic pathways, biosynthesis of secondary metabolites, and microbial metabolism. Hydroxybutyryl-CoA dehydrogenase is also referred to as 3-hydroxyacyl-CoA dehydrogenase, beta-hydroxyacyl dehydrogenase, beta-keto-reductase, 3-keto reductase, 3-hydroxyacyl coenzyme A dehydrogenase, beta-hydroxyacyl-coenzyme A synthetase, beta-hydroxyacylcoenzyme A dehydrogenase, beta-hydroxybutyrylcoenzyme A dehydrogenase, 3-hydroxyacetyl-coenzyme A dehydrogenase, L-3-hydroxyacyl coenzyme A dehydrogenase, L-3-hydroxyacyl CoA dehydrogenase, beta-hydroxyacyl CoA dehydrogenase, 3beta-hydroxyacyl coenzyme A dehydrogenase, 3-hydroxybutyryl-CoA dehydrogenase, beta-ketoacyl-CoA reductase, beta-hydroxy acid dehydrogenase, 3-L-hydroxyacyl-CoA dehydrogenase, 3-hydroxyisobutyryl-CoA dehydrogenase, or 1-specific DPN-linked beta-hydroxybutyric dehydrogenase. Hydroxybutyryl-CoA dehydrogenase is an oxidoreductase, acting on the CH—OH group of donors, and with NAD⁺ or NADP⁺ as an acceptor. See Hillmer et al., Biochim. Biophys. Acta 334 (1974) 12-23, Lehninger et al., Biochim. Biophys. Acta. 12 (1953) 188-202, Stern, Biochim. Biophys. Acta. 26 (1957) 448-9, and Wakil et al., J. Biol. Chem. 207 (1954) 631-8.

Enoyl-CoA hydratase (e.g., Clostridium acetobutylicum Enoyl-CoA hydratase) is an enzyme (EC 4.2.1.150) that catalyzes the conversion of 3-hydroxybutanoyl-CoA to crotonyl-CoA and is part of the central fermentation pathway where it is involved in the production of both acids and solvents. Enoyl-CoA hydratase is also referred to as short-chain-enoyl-CoA hydratase, 3-hydroxybutyryl-CoA dehydratase, or crotonase, and belongs. Enoyl-CoA hydratase belongs to the following classes of enzymes: lyases, carbon-oxygen lyases, and hydro-lyases. See Waterson et al., J. Biol. Chem. 247 (1972) 5266-71 and Waterson et al., Methods. Enzymol. 71 Pt C (1981) 421-30.

Crotonyl-CoA reductase (e.g., Euglena gracilis Crotonyl-CoA reductase), also referred to as trans-2-enoyl-CoA reductase (NAD⁺), is an enzyme (EC 1.3.1.44) that catalyzes the conversion of crotonyl-CoA to butanoyl-CoA, and acts more slowly on trans-hex-2-enoyl-CoA and trans-oct-2-enoyl-CoA. Crotonyl-CoA reductase is active in the butanoate metabolism and metabolic pathways. Crotonyl-CoA reductase is a member of the following classes: oxidoreductases, acting on the CH—CH group of donors, and with NAD⁺ or NADP⁺ as an acceptor. See Inui et al., J. Biochem. (Tokyo). 100 (1986) 995-1000.

Butyraldehyde dehydrogenase is an enzyme (EC 1.2.1.57) that catalyzes the conversion of butanoyl-CoA to butyraldehyde, and acts more slowly on acetaldehydes. Butyraldehyde dehydrogenase is active in the butanoate metabolism pathway, and is an oxidoreductase, acting on the aldehyde or oxo group of donors, and with NAD⁺ or NADP⁺ as an acceptor. See Palosaari et al., J. Bacteriol. 170 (1988) 2971-6.

Alcohol dehydrogenase is an enzyme (EC 1.1.1.1) that catalyzes the conversion of butyraldehyde to n-butanol. Alcohol dehydrogenase also catalyzes the conversion of isobutyraldehyde to isobutanol, and 2-butanone to 2-butanol, as discussed below. The enzyme, a zinc protein, acts on primary or secondary alcohols or hemi-acetals with very broad specificity. Alcohol dehydrogenase is also referred to as aldehyde reductase, ADH, alcohol dehydrogenase (NAD), aliphatic alcohol dehydrogenase, ethanol dehydrogenase, NAD-dependent alcohol dehydrogenase, NAD-specific aromatic alcohol dehydrogenase, NADH-alcohol dehydrogenase, NADH-aldehyde dehydrogenase, primary alcohol dehydrogenase, or yeast alcohol dehydrogenase. Alcohol dehydrogenase is an oxidoreductase, acting on the CH—OH group of donors, with NAD⁺ or NADP⁺ as an acceptor, and is active in the following biological pathways: glycolysis/gluconeogenesis, fatty acid degradation, glycine, serine and threonine metabolism, tyrosine metabolism, alpha-linolenic acid metabolism, chloroalkane and chloroalene degradation, naphthalene degradation, retinol metabolism, metabolism of xenobiotics by cytochrome P450, drug metabolism, metabolic pathways, biosynthesis of secondary metabolites, and microbial metabolism. See Branden et al., in: Boyer, P. D. (Ed.), The Enzymes, 3rd ed., vol. 11, Academic Press, New York, 1975, p. 103-190, Jornvall, Eur. J. Biochem. 72 (1977) 443-52, Negelein et al., Biochem. Z. 293 (1937) 351-389, Sund et al., and Theorell, Adv. Enzymol. Relat. Subj. Biochem. 20 (1958) 31-49.

Stoichiometry for production of n-butanol from glucose is shown below.

glucose+2ADP+2Pᵢ+2H⁺→n-butanol+2CO2+2ATP+3H₂O

ATP hydrolysis: 2ATP+2H₂O→2ADP+2Pᵢ+2H⁺

Net: glucose→n-butanol+2CO₂+3H₂O

### Isobutanol Biosynthetic Pathway

Some aspects of the present disclosure relate specifically to the production of isobutanol.

Enzymes of the isobutanol biosynthetic pathway include enzymes of the glycolytic pathway (e.g., glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase; Table 1) as well as acetolactate synthase, ketol-acid reductoisomerase, dihydroxy-acid dehydratase, branched-chain-2-oxoacid decarboxylase, and alcohol dehydrogenase (Table 3). Thus, aspects of the present disclosure comprise culturing engineered cells that express at least one (e.g., 1, 2, 3, 4, 5, 6, 7, 8, 9, 10, 11, 12, 13, 14, or 15) enzyme of the isobutanol biosynthetic pathway selected from the group consisting of glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, ketol-acid reductoisomerase, dihydroxy-acid dehydratase, branched-chain-2-oxoacid decarboxylase, and alcohol dehydrogenase. In some embodiments, methods comprise culturing engineered cells that express at least one (e.g., 1, 2, 3, 4, 5, 6, 7, 8, 9, or 10) of glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase. In some embodiments, methods comprise culturing engineered cells that express at least one (e.g., 1, 2, 3, 4, or 5) of acetolactate synthase, ketol-acid reductoisomerase, dihydroxy-acid dehydratase, branched-chain-2-oxoacid decarboxylase, and alcohol dehydrogenase.

TABLE 3
Enzymes Used for the Conversion of Pyruvate to Isobutanol

| EC | | | |
|---|---|---|---|
| Number | Activity | Substrate | Product |
| 2.2.1.6 | acetolactate synthase | pyruvate | (S)-2-acetolactate |
| 1.1.1.86 | ketol-acid reductoisomerase | (S)-2-acetolactate | (R)-2,3-dihydroxy-3- |
| | | | methylbutanoate |
| 4.2.1.9 | dihydroxy-acid dehydratase | (R)-2,3-dihydroxy-3- | 3-methyl-2-oxobutanoate |
| | | methylbutanoate | |
| 4.1.1.72 | branched-chain-2-oxoacid | 3-methyl-2-oxobutanoate | isobutyraldehyde |
| | decarboxylase | | |
| 1.1.1.1 | alcohol dehydrogenase | isobutyraldehyde | isobutanol |

Acetolactate synthase is an enzyme (EC 2.2.1.6) that catalyzes the conversion of pyruvate to (S)-2-acetolactate and is active in the following biological pathways: valine, leucine and isoleucine biosynthesis, butanoate metabolism, C5-branched dibasic acid metabolism, pantothenate and CoA biosynthesis, metabolic pathways, and biosynthesis of secondary metabolites. Acetolactate synthase is also referred to as alpha-acetohydroxy acid synthetase, alpha-acetohydroxyacid synthase, alpha-acetolactate synthase, alpha-acetolactate synthetase, acetohydroxy acid synthetase, acetohydroxyacid synthase, acetolactate pyruvate-lyase (carboxylating), or acetolactic synthetase. Acetolactate synthase belongs to the following classes of enzymes: transferases, transferring aldehyde or ketonic groups, and transketolases and transaldolases. See Bauerle et al., Biochim. Biophys. Acta. 92 (1964) 142-9, Huseby et al., Eur. J. Biochem. 20 (1971) 209-14, Stormer et al., Eur. J. Biochem. 10 (1969) 251-60, and Barak et al., J. Bacteriol. 169 (1987) 3750-6, each of which is incorporated by reference herein.

Ketol-acid reductoisomerase is an enzyme (EC 1.1.1.86) that catalyzes the conversion of (S)-2-acetolactate to (R)-2,3-dihydroxy-3-methylbutanoate as well as the reduction of 2-aceto-2-hydroxybutanoate to 2,3-dihydroxy-3-methylpentanoate, and is active in the following biological pathways: valine, leucine, and isoleucine biosynthesis, pantothenate and CoA biosynthesis, metabolic pathways, and the biosynthesis of secondary metabolites. Ketol-acid reductoisomerase is also referred to as dihydroxyisovalerate dehydrogenase (isomerizing), acetohydroxy acid isomeroreductase, ketol acid reductoisomerase, alpha-keto-beta-hydroxyacyl reductoisomerase, 2-hydroxy-3-keto acid reductoisomerase, acetohydroxy acid reductoisomerase, acetolactate reductoisomerase, dihydroxyisovalerate (isomerizing) dehydrogenase, isomeroreductase, and reductoisomerase. Ketol-acid reductoisomerase belongs to the following classes of enzymes: oxidoreductases, acting on the CH—OH group of donors, with NAD+ or NADP+ as an acceptor. See Arfin et al., J. Biol. Chem. 244 (1969) 1118-27, Hill et al., Bioorg. Chem. 8 (1979) 175-189, Kiritani et al., J. Biol. Chem. 241 (1966) 2047-2051, and Satyanarayana et al., Biochim. Biophys. Acta. 110 (1965) 380-8, each of which is incorporated by reference herein.

Dihydroxy-acid dehydratase is an enzyme (EC 4.2.1.9) that catalyzes the conversion of (R)-2,3-dihydroxy-3-methylbutanoate to 3-methyl-2-oxobutanoate, and is active in the following pathways: valine, leucine and isoleucine biosynthesis, pantothenate and CoA biosynthesis, metabolic pathways, and the biosynthesis of secondary metabolites. Dihydroxy-acid dehydratase is also referred to as acetohydroxyacid dehydratase, alpha,beta-dihydroxyacid dehydratase, 2,3-dihydroxyisovalerate dehydratase, alpha,beta-dihydroxyisovalerate dehydratase, dihydroxy acid dehydrase, DHAD, or 2,3-dihydroxy-acid hydro-lyase. Dihydroxy-acid dehydratase is part of the following classes of enzymes: lyases, carbon-oxygen lyases, and hydro-lyases. See Kanamori et al., J. Biol. Chem. 238 (1963) 998-1005, and Myers, J. Biol. Chem. 236 (1961) 1414-8, each of which is incorporated by reference herein.

Branched-chain-2-oxoacid decarboxylase is an enzyme (EC 4.1.1.72) that catalyzes the conversion of 3-methyl-2-oxobutanoate to isobutyraldehyde. The enzyme acts on various 2-oxo acids, showing a high affinity for branched-chain substrates. Branched-chain-2-oxoacid decarboxylase is also referred to as branched-chain oxo acid decarboxylase, branched-chain alpha-keto acid decarboxylase, branched-chain keto acid decarboxylase, BCKA, or (3S)-3-methyl-2-oxopentanoate carboxy-lyase. Branched-chain-2-oxoacid decarboxylase belongs to the following classes of enzymes: lyases, carbon-carbon lyases, and carboxy-lyases. See Oku et al., J. Biol. Chem. 263 (1988) 18386-96, each of which is incorporated by reference herein.

Alcohol dehydrogenase is an enzyme (EC 1.1.1.1) that catalyzes the conversion of isobutyraldehyde to isobutanol. Alcohol dehydrogenase also catalyzes the conversion of butyraldehyde to n-butanol, as discussed above.

Engineered cells may express any single enzyme, or any combination of enzymes, of an isobutanol biosynthetic pathway.

For example, cells that express enzymes of an isobutanol pathway may express only acetolactate synthase, ketol-acid reductoisomerase, dihydroxy-acid dehydratase, branched-chain-2-oxoacid decarboxylase, or alcohol dehydrogenase.

Alternatively, cells that express enzymes of an isobutanol biosynthetic pathway may express any combination of two or more enzymes. For example, cells that express enzymes of an isobutanol biosynthetic pathway may express: acetolactate synthase and ketol-acid reductoisomerase; ketol-acid reductoisomerase and dihydroxy-acid dehydratase; dihydroxy-acid dehydratase and branched-chain-2-oxoacid decarboxylase; or branched-chain-2-oxoacid decarboxylase and alcohol dehydrogenase. In some embodiments, cells that express enzymes of an isobutanol biosynthetic pathway may express: dihydroxy-acid dehydratase and branched-chain-2-oxoacid decarboxylase.

In some embodiments, engineered cells express more than one enzyme of a biosynthetic pathway (e.g., a pyruvate, n-butanol, or isobutanol biosynthetic pathway).

Stoichiometry for production of isobutanol from glucose is shown below.

glucose+2ADP+2Pi+2H⁺→isobutanol+2CO₂+2ATP+3H₂O

ATP hydrolysis: 2ATP+2H₂O→2ADP+2Pi+2H⁺

Net: glucose→isobutanol+2CO₂+3H₂O

### 2-Butanol Biosynthetic Pathway

Some aspects of the present disclosure relate specifically to the production of 2-butanol.

Enzymes of the 2-butanol biosynthetic pathway include enzymes of the glycolytic pathway (e.g., glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase; Table 1) as well as acetolactate synthase, acetolactate decarboxylase, diacetyl reductase, diol dehydratase or glycerol dehydratase, and butanol dehydrogenase (Table 3). Thus, aspects of the present disclosure comprise culturing engineered cells that express at least one (e.g., 1, 2, 3, 4, 5, 6, 7, 8, 9, 10, 11, 12, 13, 14, or 15) enzyme of the 2-butanol biosynthetic pathway selected from the group consisting of glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, acetolactate decarboxylase, diacetyl reductase, diol dehydratase or glycerol dehydratase, and butanol dehydrogenase. In some embodiments, methods comprise culturing engineered cells that express at least one (e.g., 1, 2, 3, 4, 5, 6, 7, 8, 9, or 10) of glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase. In some embodiments, methods comprise culturing engineered cells that express at least one (e.g., 1, 2, 3, 4, or 5) of acetolactate synthase, acetolactate decarboxylase, diacetyl reductase, diol dehydratase or glycerol dehydratase, and butanol dehydrogenase.

TABLE 4
Enzymes Used for the Conversion of Pyruvate to 2-Butanol

| EC | | | |
|---|---|---|---|
| Number | Activity | Substrate | Product |
| 2.2.1.6 | acetolactate synthase | pyruvate | acetolactate |
| 4.1.1.5 | acetolactate | acetolactate | acetoin |
| | decarboxylase | | |
| 1.1.1.4 | diacetyl reductase | acetoin | 2,3-butanediol |
| 4.2.1.28 or | diol dehydratase or | 2,3-butanediol | 2-butanone |
| 4.2.1.30 | glycerol dehydratase | | |
| 1.1.1.1 | alcohol (e.g., butanol) | 2-butanone | 2-butanol |
| | dehydrogenase | | |

Acetolactate synthase is an enzyme (EC 2.2.1.6) that catalyzes the conversion of pyruvate to acetolactate. Acetolactate synthase also catalyzes the conversion of pyruvate to (S)-2-acetolactate, as discussed above.

Acetolactate decarboxylase (EC 4.1.1.5) is an enzyme that catalyzes the conversion of acetolactate to acetoin, and is active in butanoate metabolism and C5-branched dibasic acid metabolism. Acetolactate decarboxylase is also referred to as alpha-acetolactate decarboxylase, (S)-2-hydroxy-2-methyl-3-oxobutanoate carboxy-lyase, (S)-2-hydroxy-2-methyl-3-oxobutanoate carboxy-lyase [(R)-2-acetoin-forming], or (S)-2-hydroxy-2-methyl-3-oxobutanoate carboxy-lyase [(3R)-3-hydroxybutan-2-one-forming]. Acetolactate decarboxylase is part of the following classes of enzymes: lyases, carbon-carbon lyases, and carboxy-lyases. See Hill et al., Bioorg. Chem. 8 (1979) 175-189; and Stormer et al., J. Biol. Chem. 242 (1967) 1756-9, each of which is incorporated by reference herein.

Diacetyl reductase (EC 1.1.1.4) is an oxidoreductase enzyme that catalyzes the conversion of acetoin to 2,3-butanediol, and is active in butanoate metabolism. Diacetyl reductase is also referred to as (R,R)-butanediol dehydrogenase, butyleneglycol dehydrogenase, D-butanediol dehydrogenase, D-(−)-butanediol dehydrogenase, butylene glycol dehydrogenase, D-aminopropanol dehydrogenase, 1-amino-2-propanol dehydrogenase, 2,3-butanediol dehydrogenase, D-1-amino-2-propanol dehydrogenase, (R)-diacetyl reductase, (R)-2,3-butanediol dehydrogenase, D-1-amino-2-propanol:NAD+ oxidoreductase, 1-amino-2-propanol oxidoreductase, or aminopropanol oxidoreductase. Diacetyl reductase See Strecker et al., J. Biol. Chem. 211 (1954) 263-70; and Taylor, et al., Biophys. Acta. 39 (1960) 448-57, each of which is incorporated by reference herein.

Diol dehydratase (EC 4.2.1.28) is an enzyme that catalyzes the conversion of 2,3-butanediol to 2-butanone, and is active in propanoate metabolism. Diol dehydratase is also referred to as propanediol dehydratase, meso-2,3-butanediol dehydrase, diol dehydratase, DL-1,2-propanediol hydro-lyase, diol dehydrase, adenosylcobalamin-dependent propanediol dehydrase, coenzyme B12-dependent diol dehydrase, 1,2-propanediol dehydratase, or propane-1,2-diol hydro-lyase. Diol dehydratase is part of the following classes of enzymes: lyases, carbon-oxygen lyases, and hydro-lyases. See Abeles et al., J. Biol. Chem. 236 (1961) 2347-50; Forage et al., J. Bacteriol. 149 (1982) 413-9; and Lee et al., J. Biol. Chem. 238 (1963) 2367-73, each of which is incorporated by reference herein.

Glycerol dehydratase (EC 4.2.1.30) is an enzyme that catalyzes the conversion of 2,3-butanediol to 2-butanone, and is active in glycerolipid metabolism. Glycerol dehydratase is also referred to as glycerol dehydrase or glycerol hydro-lyase. Glycerol dehydratase is part of the following classes of enzymes: lyases, carbon-oxygen lyases, and hydro-lyases. See Forage et al., J. Bacteriol. 149 (1982) 413-9; and Schneider et al., J. Biol. Chem. 245 (1970) 3388-96; Schneider et al., Acta. Biochim. Pol. 13 (1966) 311-28; and Smiley et al., Arch. Biochem. Biophys. 97 (1962) 538-43, each of which is incorporated by reference herein.

Alcohol dehydrogenase is an enzyme (EC 1.1.1.1) that catalyzes the conversion of 2-butanone to 2-butanol. Alcohol dehydrogenase also catalyzes the conversion of butyraldehyde to n-butanol, and isobutyraldehyde to isobutanol, as discussed above.

### Engineered Cells and Nucleic Acids

“Engineered cells” of the present disclosure are cells that comprise at least one engineered (e.g., recombinant or synthetic) nucleic acid, or are otherwise modified such that they are structurally and/or functionally distinct from their naturally-occurring counterparts. In some embodiments, an engineered cell comprises at least 1, at least 2, at least 3, at least 4, at least 5, at least 6, at least 7, at least 8, at least 9, or at least 10 engineered nucleic acids. In some embodiments, an engineered cell comprises 2 to 5, 2 to 10, or 2 to 20 engineered nucleic acids. In some embodiments, an engineered cell comprises 1, 2, 3, 4, 5, 6, 7, 8, 9, 10, 11, 12, 13, 14, 15, 16, 17, 18, 19, or 20 engineered nucleic acids.

In some embodiments, a culture of “engineered cells” contains a homogenous population or a heterogeneous population of cells. For example, a culture of engineered cells may contain more than one type of cell, each type of cell expressing at least one enzyme of a butanol biosynthetic pathway.

An enzyme of a biosynthetic pathway that is expressed by engineered cells of the present disclosure may be encoded by an endogenous nucleic acid or an engineered nucleic acid. For example, enzymes of the glycolytic pathway may be expressed by an endogenous nucleic acid, while additional enzymes (or at least some of the enzymes) of a butanol biosynthetic pathway may be expressed by an engineered nucleic acid. In some embodiments, enzymes of the glycolytic pathway are expressed chromosomally, while enzymes (or at least some of the enzymes) of a butanol biosynthetic pathway are expressed episomally.

The term “nucleic acid” refers to at least two nucleotides covalently linked together, and in some instances, may contain phosphodiester bonds (e.g., a phosphodiester “backbone”). Nucleic acids (e.g., components, or portions, of nucleic acids) may be naturally occurring or engineered. “Naturally occurring” nucleic acids are present in a cell that exists in nature in the absence of human intervention. “Engineered nucleic acids” include recombinant nucleic acids and synthetic nucleic acids. A “recombinant nucleic acid” refers to a molecule that is constructed by joining nucleic acid molecules (e.g., from the same species or from different species) and, typically, can replicate in a living cell. A “synthetic nucleic acid” refers to a molecule that is chemically or by other means synthesized or amplified, including those that are chemically or otherwise modified but can base pair with naturally occurring nucleic acid molecules. Recombinant and synthetic nucleic acids also include those molecules that result from the replication of either of the foregoing. It should be understood that engineered nucleic acids may contain portions of nucleic acids that are naturally occurring, but as a whole, engineered nucleic acids do not occur naturally and require human intervention. In some embodiments, a nucleic acid encoding an enzyme of a butanol biosynthetic pathway is a recombinant nucleic acid or a synthetic nucleic acid. In other embodiments, a nucleic acid encoding an enzyme of a butanol biosynthetic pathway is naturally occurring.

An engineered nucleic acid encoding an enzyme of a biosynthetic pathway, as provided herein, is operably linked to a “promoter,” which is a control region of a nucleic acid at which initiation and rate of transcription of the remainder of a nucleic acid are controlled. A promoter drives expression or drives transcription of the nucleic acid that it regulates. A promoter is considered to be “operably linked” when it is in a correct functional location and orientation in relation to the nucleic acid it regulates to control (“drive”) transcriptional initiation and/or expression of that nucleic acid.

Engineered nucleic acids of the present disclosure may contain a constitutive promoter or an inducible promoter. A “constitutive promoter” refers to a promoter that is constantly active in a cell. An “inducible promoter” refers to a promoter that initiates or enhances transcriptional activity when in the presence of, influenced by, or contacted by an inducer or inducing agent. Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters, steroid-regulated promoters, metal-regulated promoters, pathogenesis-regulated promoters, temperature/heat-inducible, and light-regulated promoters. In some embodiments, a nucleic acid encoding an enzyme of a butanol biosynthetic pathway is operably linked to an inducible promoter.

Enzymes of the present disclosure may be encoded by nucleic acids that are located genomically (referred to as a “genomically-located nucleic acid”) or are located episomally (referred to as an “episomally-located nucleic acid”). A nucleic acid that is located genomically in a cell is a nucleic acid that is located in the genome of the cell. A nucleic acid that is located episomally in a cell is a nucleic acid that is located on an autonomously-replicating episome in the cell, such as a plasmid. Genomically-located nucleic acids and episomally-located nucleic acids may be endogenous (e.g., originating from within the cell) to the cell or exogenous to the cell (e.g., originating from outside the cell). Typically, exogenous nucleic acids are engineered nucleic acids (e.g., recombinant or synthetic).

Engineered nucleic acids may be introduced into host cells using any means known in the art, including, without limitation, transformation, transfection (e.g., chemical (e.g., calcium phosphate, cationic polymers, or liposomes) or non-chemical (e.g., electroporation, sonoporation, impalefection, optical transfection, hydro dynamic)), and transduction (e.g., viral transduction).

Engineered cells, in some embodiments, express selectable markers. Selectable markers are typically used to select engineered cells that have taken up and expressed an engineered nucleic acid following transfection of the cell (or following other procedure used to introduce foreign nucleic acid into the cell). Thus, a nucleic acid encoding an enzyme of a biosynthetic pathway may also encode a selectable marker. Examples of selectable markers include, without limitation, genes encoding proteins that increase or decrease either resistance or sensitivity to antibiotics (e.g., ampicillin resistance genes, kanamycin resistance genes, neomycin resistance genes, tetracycline resistance genes and chloramphenicol resistance genes) or other compounds. Other selectable markers may be used in accordance with the present disclosure.

An engineered cell “expresses” an enzyme if the enzyme, encoded by a nucleic acid (e.g., an engineered nucleic acid), is produced in the cell. It is well known in the art that gene expression refers to the process by which genetic instructions in the form of a nucleic acid are used to synthesize a product, such as a protein (e.g., an enzyme).

Engineered cells may express any single enzyme, or any combination of enzymes, of a biosynthetic pathway. In some embodiments, engineered cells express all the enzymes of a butanol biosynthetic pathway. For example, an engineered cell may express all the enzymes required to produce butanol (e.g., n-butanol, isobutanol, or 2-butanol) from glucose. The enzymes expressed by an engineered cell may be encoded by naturally-occurring nucleic acids, engineered nucleic acids, or a combination thereof (e.g., at least one encoded by a naturally-occurring nucleic acid, at least one encoded by an engineered nucleic acid).

Enzymes encoded by an engineered nucleic acid may be referred to as “engineered enzymes.” Thus, in some embodiments, an engineered cell expresses at least 1, at least 2, at least 3, at least 4, at least 5, at least 6, at least 7, at least 8, at least 9, or at least 10 engineered enzymes. In some embodiments, an engineered cell expresses 2 to 5, 2 to 10, or 2 to 20 engineered enzymes. In some embodiments, an engineered cell expresses 1, 2, 3, 4, 5, 6, 7, 8, 9, 10, 11, 12, 13, 14, 15, 16, 17, 18, 19, or 20 engineered enzymes.

In some embodiments, enzymes of the present disclosure may be engineered to contain a protease-recognition sequence or a periplasmic-targeting sequence, as provided herein.

Enzymes encoded by a naturally-occurring nucleic acid may be referred to as “endogenous enzymes.” Thus, in some embodiments, an engineered cell expresses at least 1, at least 2, at least 3, at least 4, at least 5, at least 6, at least 7, at least 8, at least 9, or at least 10 endogenous enzymes. In some embodiments, an engineered cell expresses 2 to 5, 2 to 10, or 2 to 20 endogenous enzymes. In some embodiments, an engineered cell expresses 1, 2, 3, 4, 5, 6, 7, 8, 9, 10, 11, 12, 13, 14, 15, 16, 17, 18, 19, or 20 endogenous enzymes.

In some embodiments, an engineered cell expresses endogenous enzymes of the glycolytic pathway and expresses at least 1 (e.g., 1, 2, 3, 4, 5, 6 or 7) of the following engineered enzymes of the n-butanol biosynthetic pathway: pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase, which are encoded by engineered nucleic acids.

In some embodiments, an engineered cell expresses endogenous enzymes of the glycolytic pathway and expresses at least 1 (e.g., 1, 2, 3, 4, or 5) of the following engineered enzymes of the isobutanol biosynthetic pathway: acetolactate synthase, ketol-acid reductoisomerase, dihydroxy-acid dehydratase, branched-chain-2-oxoacid decarboxylase, and alcohol dehydrogenase, which are encoded by engineered nucleic acids.

In some embodiments, an engineered cell expresses endogenous enzymes of the glycolytic pathway and expresses at least 1 (e.g., 1, 2, 3, 4, or 5) of the following engineered enzymes of the 2-butanol biosynthetic pathway: acetolactate synthase, acetolactate decarboxylase, diacetyl reductase, diol dehydratase or glycerol dehydratase, and butanol dehydrogenase, which are encoded by engineered nucleic acids.

Engineered cells may be prokaryotic cells or eukaryotic cells. In some embodiments, engineered cells are bacterial cells, yeast cells, insect cells, mammalian cells, or other types of cells.

Engineered bacterial cells of the present disclosure include, without limitation, engineered Escherichia spp., Streptomyces spp., Zymomonas spp., Acetobacter spp., Citrobacter spp., Synechocystis spp., Rhizobium spp., Clostridium spp., Corynebacterium spp., Streptococcus spp., Xanthomonas spp., Lactobacillus spp., Lactococcus spp., Bacillus spp., Alcaligenes spp., Pseudomonas spp., Aeromonas spp., Azotobacter spp., Comamonas spp., Mycobacterium spp., Rhodococcus spp., Gluconobacter spp., Ralstonia spp., Acidithiobacillus spp., Microlunatus spp., Geobacter spp., Geobacillus spp., Arthrobacter spp., Flavobacterium spp., Serratia spp., Saccharopolyspora spp., Thermus spp., Stenotrophomonas spp., Chromobacterium spp., Sinorhizobium spp., Saccharopolyspora spp., Agrobacterium spp., and Pantoea spp.

Engineered yeast cells of the present disclosure include, without limitation, engineered Saccharomyces spp., Schizosaccharomyces, Hansenula, Candida, Kluyveromyces, Yarrowia and Pichia

In some embodiments, engineered cells of the present disclosure are engineered Escherichia coli cells, Bacillus subtilis cells, Pseudomonas putida cells, Saccharomyces cerevisae cells, or Lactobacillus brevis cells. In some embodiments, engineered cells of the present disclosure are engineered Escherichia coli cells

### Cell Culture

Typically, engineered cells expressing enzymes of a biosynthetic pathway are cultured. “Culturing” refers to the process by which cells are grown under controlled conditions, typically outside of their natural environment. For example, engineered cells, such as engineered bacterial cells, may be grown as a cell suspension in liquid nutrient broth, also referred to as liquid “culture medium.”

Examples of commonly used bacterial Escherichia coli growth media include, without limitation, LB (Luria Bertani) Miller broth (1% NaCl): 1% peptone, 0.5% yeast extract, and 1% NaCl; LB (Luria Bertani) Lennox Broth (0.5% NaCl): 1% peptone, 0.5% yeast extract, and 0.5% NaCl; SOB medium (Super Optimal Broth): 2% peptone, 0.5% Yeast extract, 10 mM NaCl, 2.5 mM KCl, 10 mM MgCl₂, 10 mM MgSO₄; SOC medium (Super Optimal broth with Catabolic repressor): SOB+20 mM glucose; 2×YT broth (2× Yeast extract and Tryptone): 1.6% peptone, 1% yeast extract, and 0.5% NaCl; TB (Terrific Broth) medium: 1.2% peptone, 2.4% yeast extract, 72 mM K₂HPO₄, 17 mM KH₂PO₄ and 0.4% glycerol; and SB (Super Broth) medium: 3.2% peptone, 2% yeast extract, and 0.5% NaCl.

Examples of high density bacterial Escherichia coli growth media include DNAGro™ medium, ProGro™ medium, AutoX™ medium, DetoX™ medium, InduX™ medium, and SecPro™ medium.

In some embodiments, engineered cells are cultured under conditions that result in expression of enzymes of a biosynthetic pathway. Such culture conditions may depend on the particular enzymes being expressed and the desired amount of the enzymes.

In some embodiments, engineered cells are cultured at a temperature of 30° C. to 40° C. For example, engineered cells may be cultured at a temperature of 30° C., 31° C., 32° C., 33° C., 34° C., 35° C., 36° C., 37° C., 38° C., 39° C. or 40° C. Typically, engineered cells, such as engineered bacterial cells, are cultured at a temperature of 37° C.

In some embodiments, engineered cells are cultured for a period of time of 12 hours to 72 hours, or more. For example, engineered cells may be cultured for a period of time of 12, 18, 24, 30, 36, 42, 48, 54, 60, 66, or 72 hours. Typically, engineered cells, such as engineered bacterial cells, are cultured for a period of time of 12 to 24 hours. In some embodiments, engineered cells are cultured for 12 to 24 hours at a temperature of 37° C.

In some embodiments, engineered cells expressing enzymes of a biosynthetic pathway are cultured (e.g., in liquid cell culture medium) to an optical density, measured at a wavelength of 600 nm (OD600), of 5 to 25. In some embodiments, engineered cells are cultured to an OD600 of 5, 10, 15, 20, or 25.

In some embodiments, engineered cells are cultured to a density of 1×10⁴ to 1×10⁸ viable cells/ml cell culture medium. In some embodiments, engineered cells are cultured to a density of 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, or 1×10⁸ viable cells/ml. In some embodiments, engineered cells are cultured to a density of 2×10⁵ to 3×10⁷ viable cells/ml.

In some embodiments, engineered cells are cultured in a bioreactor. A bioreactor refers simply to a container in which cells are cultured, such as a culture flask, a dish, or a bag that may be single-use (disposable), autoclavable, or sterilizable. The bioreactor may be made of glass, or it may be polymer-based, or it may be made of other materials.

Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor may be a batch or continuous processes and will depend on the engineered cells being cultured. A bioreactor is continuous when the feed and product streams are continuously being fed and withdrawn from the system. A batch bioreactor may have a continuous recirculating flow, but no continuous feeding of nutrient or product harvest. For intermittent-harvest and fedbatch (or batch fed) cultures, cells are inoculated at a lower viable cell density in a medium that is similar in composition to a batch medium. Cells are allowed to grow exponentially with essentially no external manipulation until nutrients are somewhat depleted and cells are approaching stationary growth phase. At this point, for an intermittent harvest batch-fed process, a portion of the cells and product may be harvested, and the removed culture medium is replenished with fresh medium. This process may be repeated several times. For production of recombinant proteins and antibodies, a fedbatch process may be used. While cells are growing exponentially, but nutrients are becoming depleted, concentrated feed medium (e.g., 10-15 times concentrated basal medium) is added either continuously or intermittently to supply additional nutrients, allowing for further increase in cell concentration and the length of the production phase. Fresh medium may be added proportionally to cell concentration without removal of culture medium (broth). To accommodate the addition of medium, a fedbatch culture is started in a volume much lower that the full capacity of the bioreactor (e.g., approximately 40% to 50% of the maximum volume).

Some methods of the present disclosure are directed to large-scale production of pyruvate, n-butanol, isobutanol, or 2-butanol. For large-scale production methods, engineered cells (e.g., that express enzymes of a pyruvate, n-butanol, isobutanol, or 2-butanol biosynthetic pathway) may be grown in liquid culture medium in a volume of 5 liters (L) to 50 L, or more. In some embodiments, engineered cells may be grown in liquid culture medium in a volume of greater than (or equal to) 10 L. In some embodiments, engineered cells are grown in liquid culture medium in a volume of 5 L, 10 L, 15 L, 20 L, 25 L, 30 L, 35 L, 40 L, 45 L, or 50 L, or more. In some embodiments, engineered cells may be grown in liquid culture medium in a volume of 5 L to 10 L, 5 L to 15 L, 5 L to 20 L, 5 L to 25 L, 5 L to 30 L, 5 L to 35 L, 5 L to 40 L, 5 L to 45 L, 10 L to 15 L, 10 L to 20 L, 10 L to 25 L, 20 L to 30 L, 10 L to 35 L, 10 L to 40 L, 10 L to 45 L, 10 L to 50 L, 15 L to 20 L, 15 L to 25 L, 15 L to 30 L, 15 L to 35 L, 15 L to 40 L, 15 L to 45 L, or 15 to 50 L.

In some embodiments, butanol (e.g., n-butanol, isobutanol, or 2-butanol) is produced at a concentration of greater than (or equal to) 2% v/v. For example, butanol may be produced at a concentration of 2% v/v to 25% v/v. In some embodiments, butanol is produced at a concentration of 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, or more. In some embodiments, butanol is produced at a concentration of at least 5%, at least 10%, at least 15%, at least 20%, or at least 25%. In some embodiments, butanol is produced at a concentration of 2% v/v to 5% v/v, 2% v/v to 10% v/v, 2% v/v to 15% v/v, 2% v/v to 20% v/v, 2% v/v to 25% v/v, 5% v/v to 10% v/v, 5% v/v to 15% v/v, 5% v/v to 20% v/v, or 5% v/v to 25% v/v.

In some embodiments, butanol (e.g., n-butanol, isobutanol, or 2-butanol) is produced with a titer of greater than (or equal to) 2 g/L. For example, butanol may be produced with a titer of 2 g/L to 20 g/L. In some embodiments, butanol is produced with a titer of 2 g/L, 3 g/L, 4 g/L, 5 g/L, 6 g/L, 7 g/L, 8 g/L, 9 g/L, 10 g/L, 15 g/L, or 20 g/L. In some embodiments, butanol is produced with a titer of 2 g/L to 5 g/L, 2 g/L to 6 g/L, 2 g/L to 7 g/L, 2 g/L to 8 g/L, 2 g/L to 9 g/L, 2 g/L to 10 g/L, 3 g/L to 5 g/L, 3 g/L to 6 g/L, 3 g/L to 7 g/L, 3 g/L to 8 g/L, 3 g/L to 9 g/L, 3 g/L to 10 g/L, 4 g/L to 5 g/L, 4 g/L to 6 g/L, 4 g/L to 7 g/L, 4 g/L to 8 g/L, 4 g/L to 9 g/L, 4 g/L to 10 g/L, 5 g/L to 6 g/L, 5 g/L to 7 g/L, 5 g/L to 8 g/L, 5 g/L to 9 g/L, or 5 g/L to 10 g/L.

In some embodiments, butanol (e.g., n-butanol, isobutanol, or 2-butanol) is produced at a yield of greater than (or equal to) 0.25 g/g glucose. For example, butanol may be produced at a yield of 0.25 g/g to 0.4 g/g glucose. In some embodiments, butanol is produced at a yield of 0.25 g/g, 0.30 g/g, 0.35 g/g, 0.40 g/g, In some embodiments, butanol is produced at a yield of 0.25 g/g.

In some embodiments, butanol (e.g., n-butanol, isobutanol, or 2-butanol) is produced with a productivity of greater than (or equal to) 1.0 g/Lh. For example, butanol may be produced with a productivity of 1.0 g/Lh to 20 g/Lh. In some embodiments, butanol is produced with a productivity 1 g/Lh, 2 g/Lh, 3 g/Lh, 4 g/Lh, 5 g/Lh, 6 g/Lh, 7 g/Lh, 8 g/Lh, 9 g/Lh, 10 g/Lh, 11 g/Lh, 12 g/Lh, 13 g/Lh, 14 g/Lh, 15 g/Lh, 16 g/Lh, 17 g/Lh, 17 g/Lh, 18 g/Lh, or 20 g/Lh. In some embodiments, butanol is produced at a yield of 1 g/Lh to 5 g/Lh, 2 g/Lh to 5 g/Lh, 1 g/Lh to 10 g/Lh, 2 g/Lh to 10 g/Lh, 1 g/Lh to 15 g/Lh, 2 g/Lh to 15 g/Lh, 2 g/Lh to 20 g/Lh, 5 g/Lh to 10 g/Lh, 5 g/Lh to 15 g/Lh, or 5 g/Lh to 20 g/Lh.

### Cell Lysates

Typically, culturing of engineered cells expressing enzymes of a biosynthetic pathway is followed by lysing the cells. “Lysing” refers to the process by which cells are broken down, for example, by viral, enzymatic, mechanical, or osmotic mechanisms. A “cell lysate” refers to a fluid containing the contents of lysed cells (e.g., lysed engineered cells), including, for example, organelles, membrane lipids, proteins, and nucleic acids. Cell lysates of the present disclosure may be produced by lysing any population of engineered cells, as provided herein.

Methods of cell lysis, referred to as “lysing,” are known in the art, any of which may be used in accordance with the present disclosure. Such cell lysis methods include, without limitation, physical lysis and chemical (e.g., detergent-based) lysis.

Cell lysis can disturb carefully controlled cellular environments, resulting in protein degradation and modification by unregulated of endogenous proteases and phosphatases. Thus, in some embodiments, protease inhibitors and/or phosphatase inhibitors may be added to lysis reagents, or these activities may be removed by gene inactivation or protease targeting.

Cell lysates of the present disclosure comprise at least one enzyme of a biosynthetic pathway. In some embodiments, a cell lysate comprises at least 1, at least 2, at least 3, at least 4, at least 5, at least 6, at least 7, at least 8, at least 9, or at least 10 enzymes of a butanol biosynthetic pathway. In some embodiments, a cell lysate comprises 2 to 5, 2 to 10, or 2 to 20 enzymes of a butanol biosynthetic pathway. In some embodiments, a cell lysate comprises 1, 2, 3, 4, 5, 6, 7, 8, 9, 10, 11, 12, 13, 14, 15, 16, 17, 18, 19, or 20 enzymes of a butanol biosynthetic pathway.

In some embodiments, cell lysates of the present disclosure containing enzymes of a biosynthetic pathway (e.g., glycolytic pathway or butanol biosynthetic pathway) are combined in a single reaction for producing butanol (or an intermediate substrate, such as pyruvate). In some embodiments, a single lysate (e.g., produced from a homogenous population of cultured engineered cells, e.g., from a single container (e.g., flask) of cultured cells) contain all the enzymes necessary to synthesize butanol from glucose. For example, a single lysate may contain all the enzymes listed in Tables 1 and 2, or all the enzymes listed in Tables 1 and 3.

In some embodiments, multiple cell lysates may be combined to form a single reaction mixture for butanol biosynthesis or for synthesis of an intermediate substrate, such as pyruvate. For example, one cell lysate comprising 1, 2, 3, 4, 5, 6, 7, 8, 9, or 10 enzymes of a glycolytic pathway or butanol biosynthetic pathway may be combined with another cell lysate comprising 1, 2, 3, 4, 5, 6, 7, 8, 9, or 10 enzymes of a glycolytic pathway or butanol biosynthetic pathway. As another example, three or more cell lysates, each comprising 1, 2, 3, 4, 5, 6, 7, 8, 9, or 10 enzymes of a glycolytic pathway or butanol biosynthetic pathway may be combined to form a single reaction mixture.

Cell lysates, in some embodiments, may be combined with at least one substrate of a biosynthetic pathway. For example, cell lysates used for the production of n-butanol may be combined with glucose, glucose-6-phosphate, fructose-6-phosphate, fructose 1,6-bisphosphate, dihydroxyacetone phosphate, glyceraldehyde 3-phosphate, 1,3-bisphosphoglycerate, 3-phospho-D-glycerate, 2-phospho-D-glycerate, phosphoenolpyruvate, pyruvate, acetyl-CoA, acetoacetyl-CoA, 3-hydroxybutanoyl-CoA, crotonyl-CoA, butanoyl-CoA, butyraldehyde, or any combination thereof. In some embodiments, cell lysates are combined with glucose.

Cell lysates used for the production of isobutanol may be combined with glucose, glucose-6-phosphate, fructose-6-phosphate, fructose 1,6-bisphosphate, dihydroxyacetone phosphate, glyceraldehyde 3-phosphate, 1,3-bisphosphoglycerate, 3-phospho-D-glycerate, 2-phospho-D-glycerate, phosphoenolpyruvate, pyruvate, (S)-2-acetolactate, (R)-2,3-dihydroxy-3-methylbutanoate, 3-methyl-2-oxobutanoate, isobutyraldehyde, or any combination thereof.

Cell lysates used for the production of 2-butanol may be combined with glucose, or another fermentable sugar, glucose-6-phosphate, fructose-6-phosphate, fructose 1,6-bisphosphate, dihydroxyacetone phosphate, glyceraldehyde 3-phosphate, 1,3-bisphosphoglycerate, 3-phospho-D-glycerate, 2-phospho-D-glycerate, phosphoenolpyruvate, pyruvate, acetolactate, acetoin, 2,3-butanediol, 2-butanone, or any combination thereof. In some embodiments, at least one cell lysate is combined with glucose during the butanol production phase. In some embodiments, glucose may be provided in a ‘fed-batch’ manner and may be required stoichiometrically one-to-one with butanol (e.g., n-butanol, isobutanol, or 2-butanol).

Cell lysates, in some embodiments, may be combined with at least one purified or partially-purified enzyme of a biosynthetic pathway. Thus, any of the enzymes listed in Tables 1-3 may be used in purified or partially purified form. “Protein purification” refers to the process, or processes, by which a protein is isolated from a mixture, such as a mixture containing cells or tissue. Protein purification methods include, for example, size exclusion chromatography, protein separation based on charge or hydrophobicity, affinity chromatography, and high performance liquid chromatography.

Cell lysates, in some embodiments, may be combined with at least one nutrient. For example, cell lysates may be combined with Na₂HPO₄, KH₂PO₄, NH₄Cl, NaCl, MgSO₄, CaCl₂. Examples of other nutrients include, without limitation, magnesium sulfate, magnesium chloride, magnesium orotate, magnesium citrate, potassium phosphate monobasic, potassium phosphate dibasic, potassium phosphate tribasic, sodium phosphate monobasic, sodium phosphate dibasic, sodium phosphate tribasic, ammonium phosphate monobasic, ammonium phosphate dibasic, ammonium sulfate, ammonium chloride, ammonium hydroxide,

Cell lysates, in some embodiments, may be combined with at least one cofactor. For example, cell lysates may be combined with adenosine diphosphate (ADP), adenosine triphosphate (ATP), nicotinamide adenine dinucleotide (NAD⁺), or other non-protein chemical compounds required for activity of an enzyme (e.g., inorganic ions and coenzymes).

In some embodiments, cell lysates (e.g., 2, 3, 4, 5, or more), each containing at least one enzyme of a butanol biosynthetic pathway, are combined in a single reaction with glucose, and are incubated under conditions that result in the production of butanol (e.g., n-butanol, isobutanol, or 2-butanol) or in intermediate substrate, such as pyruvate.

Methods of the present disclosure include incubating a (at least one) cell lysate under conditions that result in production of butanol. A cell lysate may be incubated at temperature of 4° C. to 45° C., or higher. For example, engineered cells may be incubated at a temperature of 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., or 45° C. In some embodiments, a cell lysate is incubated at a temperature of 4-6° C., 25° C., or 37° C. In some embodiments, a cell lysate is incubated at a temperature of 15° C. to 45° C.

In some embodiments, a cell lysate is incubated for a period of time of 30 minutes (min) to 48 hours (hr), or more. For example, engineered cells may be cultured for a period of time of 30 min, 45 min, 1 hr, 2 hrs, 3 hrs, 4 hrs, 5 hrs, 6 hrs, 7 hrs, 8 hrs, 9 hrs, 10 hrs, 11 hrs, 12 hrs, 18 hrs, 24 hrs, 30 hrs, 36 hrs, 42 hours, or 48 hours. In some embodiments, a cell lysate is incubated for a period of time of 2 hr to 48 hr. In some embodiments, a cell lysate is incubated for 24 hours at a temperature of 37° C.

The volume of cell lysate used for a single reaction may vary. In some embodiments, the volume of a cell lysate is 1 to 150 m³. For example, the volume of a cell lysate may be 1 m³, 5 m³, 10 m³, 15 m³, 20 m³, 25 m³, 30 m³, 35 m³, 40 m³, 45 m³, 50 m³, 55 m³, 60 m³, 65 m³, 70 m³, 75 m³, 80 m³, 85 m³, 90 m³, 95 m³, 100 m³, 105 m³, 110 m³, 115 m³, 120 m³, 125 m³, 130 m³, 135 m³, 140 m³, 145, or 150 m³. In some embodiments, the volume of a cell lysate is 25 m³ to 150 m³, 50 m³ to 150 m³, or 100 m³ to 150 m³.

### Protease Targeting

Engineered cells of the present disclosure may express (e.g., endogenously express) enzymes necessary for the health of the cells that may have a negative impact on the production of butanol (e.g., n-butanol, isobutanol, or 2-butanol). Such enzymes are referred to herein as “target enzymes.” For example, target enzymes expressed by engineered cells may compete for substrates or cofactors with an enzyme that increases the rate of precursor supplied to a butanol biosynthetic pathway. As another example, target enzymes expressed by the engineered cells may compete for substrates or cofactors with an enzyme that is a key pathway entry enzyme of a butanol biosynthetic pathway. As yet another example, target enzymes expressed by the engineered cells may compete for substrates or cofactors with an enzyme that supplies a substrate or cofactor of the isobutanol biosynthetic pathway.

To negate, or reduce, this negative impact, target enzymes can be modified to include a site-specific protease-recognition sequence in their protein sequence such that the target enzyme may be “targeted” and cleaved for inactivation during butanol production (see, e.g., U.S. Publication No. 2012/0052547 A1, published on Mar. 1, 2012; and International Publication No. WO 2015/021058 A2, published Feb. 12, 2015, each of which is incorporated by reference herein).

Cleavage of a target enzyme containing a site-specific protease-recognition sequence results from contact with a cognate site-specific protease is sequestered in the periplasm of cell (separate from the target enzyme) during the cell growth phase (e.g., as engineered cells are cultured) and is brought into contact with the target enzyme during the butanol production phase (e.g., following cell lysis to produce a cell lysate). Thus, engineered cells of the present disclosure comprise, in some embodiments, (i) an engineered nucleic acid encoding a target enzyme that negatively impacts the rate of butanol production and includes a site-specific protease-recognition sequence in the protein sequence of the target enzyme, and (ii) an engineered nucleic acid encoding a site-specific protease that cleaves the site-specific protease-recognition sequence of the target enzyme and includes a periplasmic-targeting sequence. This periplasmic-targeting sequence is responsible for sequestering the site-specific protease to the periplasmic space of the cell until the cell is lysed. Examples of periplasmic-targeting sequences are provided below.

Examples of proteases that may be used in accordance with the present disclosure include, without limitation, alanine carboxypeptidase, Armillaria mellea, astacin, bacterial leucyl aminopeptidase, cancer procoagulant, cathepsin B, clostripain, cytosol alanyl aminopeptidase, elastase, endoproteinase Brg-C, enterokinase, gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2B, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin B, venombin BB and Xaa-pro aminopeptidase.

Specific examples of target enzymes include, without limitation, pyruvate dehydrogenase, PEP carboxylase, citrate synthase, phosphate acetyltransferase, β-ketoacyl-ACP synthase III, and acetyl-CoA carboxylase. In some embodiments, the target enzyme is pyruvate dehydrogenase.

### Periplasmic Targeting

Enzymes of a butanol (or pyruvate) biosynthetic pathway may include at least one enzyme that has a negative impact on the health (e.g., viability) of a cell. To negate or reduce this negative impact, an enzyme can be modified to include a relocation sequence such that the enzyme is relocated to a cellular or extra-cellular compartment where it is not naturally located and where the enzyme does not negatively impact the health of the cell (see, e.g., Publication No. US-2011-0275116-A1, published on Nov. 10, 2011, incorporated by reference herein). For example, an enzyme of a biosynthetic pathway may be relocated to the periplasmic space of a cell.

Thus, in some embodiments, engineered cells of the present disclosure comprise at least one enzyme of a butanol (e.g., n-butanol, isobutanol, or 2-butanol) biosynthetic pathway that is linked to a periplasmic-targeting sequence. A “periplasmic-targeting sequence” is an amino acid sequence that targets to the periplasm of a cell the protein to which it is linked. A protein that is linked to a periplasmic-targeting sequence will be sequestered in the periplasm of the cell in which the protein is expressed. Any of the enzymes listed in Tables 1-3 may be linked to a periplasmic-targeting sequence.

Periplasmic-targeting sequences may be derived from the N-terminus of bacterial secretory protein, for example. The sequences vary in length from about 15 to about 70 amino acids. The primary amino acid sequences of periplasmic-targeting sequences vary, but generally have a common structure, including the following components: (i) the N-terminal part has a variable length and generally carries a net positive charge; (ii) following is a central hydrophobic core of about 6 to about 15 amino acids; and (iii) the final component includes four to six amino acids which define the cleavage site for signal peptidases.

Periplasmic-targeting sequences of the present disclosure, in some embodiments, may be derived from a protein that is secreted in a Gram negative bacterium. The secreted protein may be encoded by the bacterium, or by a bacteriophage that infects the bacterium. Examples of Gram negative bacterial sources of secreted proteins include, without limitation, members of the genera Escherichia, Pseudomonas, Klebsiella, Salmonella, Caulobacter, Methylomonas, Acetobacter, Achromobacter, Acinetobacter, Aeromonas, Agrobacterium, Alcaligenes, Azotobacter, Burkholderia, Citrobacter, Comamonas, Enterobacter, Erwinia, Rhizobium, Vibrio, and Xanthomonas.

Examples of periplasmic-targeting sequences for use in accordance with the present disclosure include, without limitation, sequences selected from the group consisting of:

(SEQ ID NO: 1)
MKIKTGARILALSALTTMMFSASALA;
(SEQ ID NO: 2)
MKQSTIALALLPLLFTPVTKA;
(SEQ ID NO: 3)
MMITLRKLPLAVAVAAGVMSAQAMA;
(SEQ ID NO: 4)
MNKKVLTLSAVMASMLFGAAAHA;
(SEQ ID NO: 5)
MKYLLPTAAAGLLLLAAQPAMA;
(SEQ ID NO: 6)
MKKIWLALAGLVLAFSASA;
(SEQ ID NO: 7)
MMTKIKLLMLIIFYLIISASAHA;
(SEQ ID NO: 8)
MKQALRVAFGFLILWASVLHA;
(SEQ ID NO: 9)
MRVLLFLLLSLFMLPAFS;
and
(SEQ ID NO: 10)
MANNDLFQASRRRFLAQLGGLTVAGMLGPSLLTPRRATA.

### Examples

### Strain Engineering

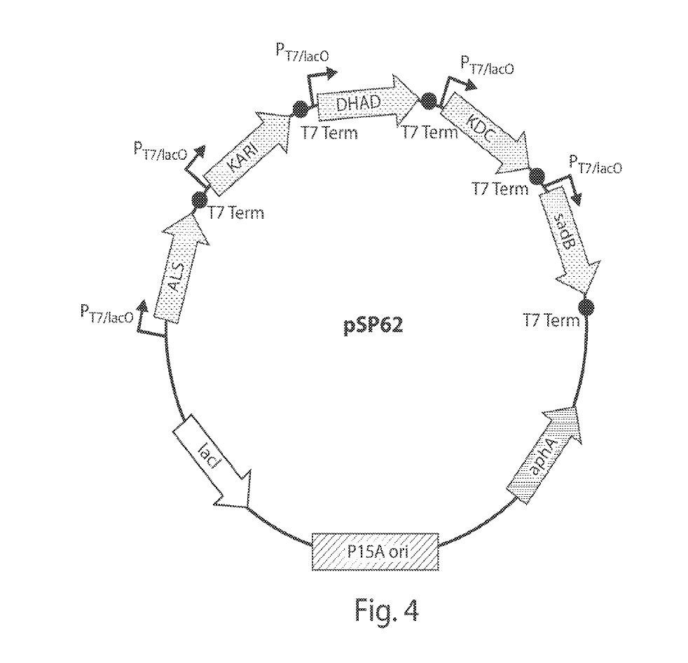

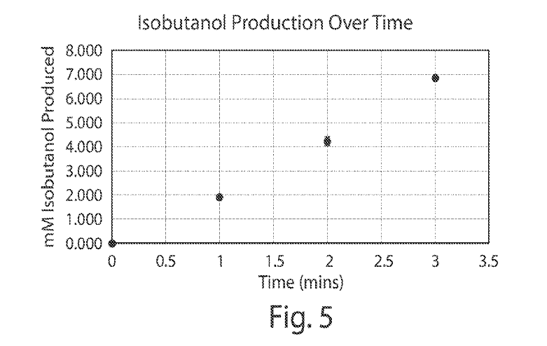

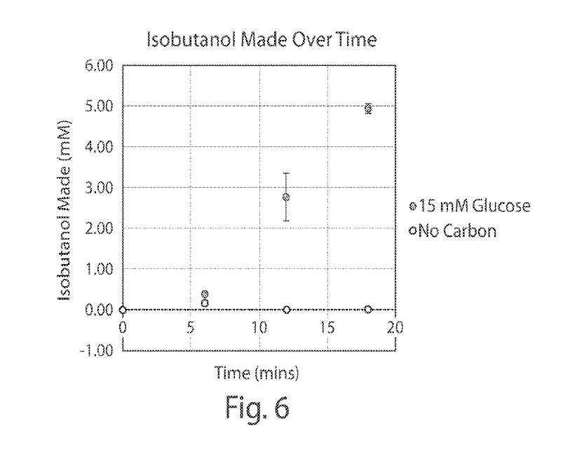

Plasmid pSP62, containing five genes encoding enzymes required for converting pyruvate to isobutanol, is shown in FIG. 4. The enzymes encoded on the plasmid are; Bacillus subtilis acetolactate synthase (ALS)¹, a mutated NADH-dependent Escherichia coli ketol-acid reductoisomerase (KARI)², Streptococcus nutans dihydroxy-acid dehydratase (DHAD)³, Lactococcus lactis α-ketoisovalerate decarboxylase (Kdc)⁴, and Achromobacter xylosoxidans butanol dehydrogenase (sadB)⁵. Each open reading frame (ORF) was synthesized in vitro and is flanked by a T7 promoter upstream and a T7 terminator downstream. The plasmid contains a lacI gene (encoding the Lac-repressor), an aphA gene (encoding neomycin resistance), and a p15A origin of replication (ori). An E. coli BL21 variant was used as the host for the resulting plasmid. This strain was made chemically competent and then transformed with pSP62. As a negative control, the same E. coli BL21 variant was transformed with pGLK063 (the empty vector). Transformants were plated on LB agar containing 50 μg/ml neomycin and incubated at 37° C. overnight.

### Cell-Free Lysate Production

E. coli BL21 (pSP62) and BL21 (pGLK063) transformants were streaked onto a LB agar with 50 μg/ml neomycin and incubated at 37° C. overnight. Colonies were then inoculated into LB medium supplemented with 50 μg/ml neomycin and grown with shaking at 250 rpm and 37° C. The culture was then diluted 1:100 into LB medium supplemented with 50 μg/ml neomycin and 0.2% glucose. These cultures were grown shaking at 37° C. until OD600-1 and then induced with 0.8 mM Isopropyl β-D-1-thiogalactopyranoside (IPTG). The cultures were grown for an additional 1 hr at 30° C. The culture was harvested by centrifugation. Cell pellets were either frozen at −80° C. or lysed immediately.

Cell pellets were resuspended in a volume of phosphate buffer and lysed by high pressure homogenization. The resulting lysates were clarified by centrifugation and the supernatants were transferred to a fresh tubes and stored at −80° C.

### Pyruvate→Isobutanol Production

Clarified cell extracts (˜40 mg/ml total protein) were thawed on ice. A solution containing 3.93 mL of clarified cell extract and 48 μL 1 M MgCl2 was preheated at 30° C. for 7 minutes. Aliquots of lysate+MgCl₂ mix were then added to 2-ml tubes with TPP, NADH and sodium pyruvate to yield reaction mixes with final concentrations of 10 mM MgCl₂, 1 mM TPP, 20 mM NADH, and 20 mM sodium pyruvate in a final volume of 600 μL. The tubes were shaken at 300 rpm at 30° C. on a Thermomixer. At varying times, duplicate reactions were quenched with 150 μL 900 mM sulfuric acid and vortexed. Quenched samples were centrifuged to remove precipitate and subsequently filtered through 0.2 μm filters.

Each reaction was analyzed by GC and HPLC to determine the concentration of isobutanol.

### Glucose→Isobutanol Production

Clarified glucose to isobutanol cell free production reactions were essentially the same as those described for the pyruvate to isobutanol reactions with the following exceptions: 15 mM glucose was added in place of 20 mM pyruvate and no NADH was added.

### Results

### Cell-Free Conversion of Pyruvate to Isobutanol

The conversion of pyruvate to isobutanol in a cell free reaction system in shown in FIG. 2. The maximum rate of isobutanol production measured was 158 mM/hr. No isobutanol was formed in the reactions containing lysate from the vector control strain.

### Cell-Free Conversion of Glucose to Isobutanol

The conversion of glucose to isobutanol in a cell free reaction system is shown in FIG. 3. The maximum rate of isobutanol production measured was 23.9 mM/hr. No isobutanol was formed in the reactions containing lysate from the vector control strain.

### REFERENCES

1. Atsumi, S., Li, Z. & Liao, J. C. Acetolactate synthase from Bacillus subtilis serves as a 2-ketoisovalerate decarboxylase for isobutanol biosynthesis in Escherichia coli. Appl. Environ. Microbiol. 75, 6306-6311 (2009).
2. Bastian, S. et al. Engineered ketol-acid reductoisomerase and alcohol dehydrogenase enable anaerobic 2-methylpropan-1-ol production at theoretical yield in Escherichia coli. Metab. Eng. 13, 345-352 (2011).
3. Kelly, K. J. & Ye, R. W. Dhad variants and methods of screening. (2014). at <http://www.google.com/patents/WO2014151190A1?cl=en>
4. De la Plaza, M., Fernandez de Palencia, P., Peláez, C. & Requena, T. Biochemical and molecular characterization of alpha-ketoisovalerate decarboxylase, an enzyme involved in the formation of aldehydes from amino acids by Lactococcus lactis. FEMS Microbiol. Lett. 238, 367-374 (2004).
5. Satagopan, S., O'Keefe, D. P. & Gude, J. Alcohol dehydrogenases (ADH) useful for fermentive production of lower alkyl alcohols. (2014). at <http://www.google.com/patents/US8765433>

### Other Embodiments

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of producing a cell lysate for producing n-butanol, the method comprising:
(a) culturing engineered cells that express at least one enzyme of a n-butanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase, wherein the cells are cultured under conditions that result in expression of enzymes; and
(b) lysing engineered cells cultured in step (a), thereby producing a cell lysate that comprises at least one enzyme of the n-butanol biosynthetic pathway.

2. The method of claim 1, wherein the engineered cells express at least 2 enzymes of the n-butanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase.

3. The method of claim 2, wherein the engineered cells express 2 to 17 enzymes of the n-butanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase.

4. The method of claim 3, wherein the engineered cells express glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase.

5. The method of any one of claims 1-4, wherein at least one enzyme of the n-butanol biosynthetic pathway expressed by the engineered cell is encoded by an endogenous nucleic acid.

6. The method of claim 5, wherein at least one enzyme encoded by an endogenous nucleic acid is selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase.

7. The method of any one of claims 1-6, wherein at least one enzyme of the n-butanol biosynthetic pathway expressed by the engineered cell is encoded by an engineered nucleic acid.

8. The method of claim 7, wherein at least one enzyme encoded by the engineered nucleic acid is selected from the group consisting of: pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase.

9. The method of any one of claims 1-8 further comprising combining the cell lysate with at least one other cell lysate that expresses at least one enzyme of the n-butanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase.

10. The method of any one of claims 1-9 further comprising combining the cell lysate with at least one purified enzyme of the n-butanol biosynthetic pathway.

11. The method of any one of claims 1-10 further comprising combining the cell lysate with glucose.

12. The method of claim 11 further comprising combining the cell lysate with at least one substance selected from the group consisting of: substrates, enzymes, nutrients, co-factors, buffers, and reducing agents.

13. The method of any one of claims 1-12 further comprising combining the cell lysate with a proton leakage agent.

14. The method of claim 13, wherein the proton leakage agent is dinitrophenol.

15. The method of any one of claims 1-14 further comprising combining the cell lysate with a phosphatase.

16. The method of any one of claims 1-15, wherein the engineered cells of step (a) further comprise:
an engineered nucleic acid encoding a target enzyme that negatively impacts the rate of n-butanol production and includes a site-specific protease-recognition sequence in the protein sequence of the target enzyme, and
an engineered nucleic acid encoding a site-specific protease that cleaves the site-specific protease-recognition sequence of the target enzyme and includes a periplasmic-targeting sequence, wherein the engineered cells are cultured under conditions that result in expression of enzymes and periplasmic sequestration of a site-specific protease that comprises a periplasmic-targeting sequence.

17. The method of claim 16, wherein target enzyme:
competes for substrates or cofactors with an enzyme that increases the rate of precursor supplied to the n-butanol biosynthetic pathway;
competes for substrates or cofactors with an enzyme that is a key pathway entry enzyme of the n-butanol biosynthetic pathway; or
competes for substrates or cofactors with an enzyme that supplies a substrate or cofactor of the n-butanol biosynthetic pathway.

18. The method of claim 17, wherein the target enzyme is selected from the group consisting of: pyruvate dehydrogenase, PEP carboxylase, citrate synthase, phosphate acetyltransferase, β-ketoacyl-ACP synthase III, and acetyl-CoA carboxylase.

19. The method of claim 17 or 18, wherein the engineered cells do not express an endogenous wild-type form of the target enzyme.

20. The method of any one of claims 16-19, wherein the site-specific protease is selected from the group consisting of: alanine carboxypeptidase, Armillaria mellea, astacin, bacterial leucyl aminopeptidase, cancer procoagulant, cathepsin B, clostripain, cytosol alanyl aminopeptidase, elastase, endoproteinase Brg-C, enterokinase, gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2B, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin B, venombin BB and Xaa-pro aminopeptidase.

21. The method of claim 20, wherein the site-specific protease is human rhinovirus 3C protease.

22. The method of any one of claims 16-21, wherein the nucleic acid encoding the site-specific protease is operably linked to an inducible promoter.

23. The method of any one of claims 1-22, wherein the engineered cells are engineered bacterial cells.

24. The method of claim 23, wherein the engineered bacterial cells are engineered Escherichia coli cells.

25. The method of any one of claims 1-24, wherein at least one of the enzymes of the n-butanol biosynthetic pathway is linked to a periplasmic-targeting sequence.

26. The method of any one of claims 16-25, wherein the periplasmic-targeting sequence is a sequence selected from the group consisting of:
(SEQ ID NO: 1)
MKIKTGARILALSALTTMMFSASALA;
(SEQ ID NO: 2)
MKQSTIALALLPLLFTPVTKA;
(SEQ ID NO: 3)
MMITLRKLPLAVAVAAGVMSAQAMA;
(SEQ ID NO: 4)
MNKKVLTLSAVMASMLFGAAAHA;
(SEQ ID NO: 5)
MKYLLPTAAAGLLLLAAQPAMA;
(SEQ ID NO: 6)
MKKIWLALAGLVLAFSASA;
(SEQ ID NO: 7)
MMTKIKLLMLIIFYLIISASAHA;
(SEQ ID NO: 8)
MKQALRVAFGFLILWASVLHA;
(SEQ ID NO: 9)
MRVLLFLLLSLFMLPAFS;
and
(SEQ ID NO: 10)
MANNDLFQASRRRFLAQLGGLTVAGMLGPSLLTPRRATA.

27. The method of any one of claims 11-26 further comprising incubating the cell lysate under conditions that result in production of n-butanol.

28. A method of producing n-butanol, comprising:
combining two or more of the cell lysates produced by the method of any one of claims 1-26 and, optionally, at least one purified enzyme of the n-butanol biosynthetic pathway; and
incubating the two or more cell lysates under conditions that result in production of n-butanol.

29. The method of claim 27 or 28 further comprising isolating the n-butanol.

30. The method of any one of claims 27-29, wherein the n-butanol is produced at a concentration of greater than 2% v/v.

31. The method of claim 30, wherein the n-butanol is produced at a concentration of greater than 5% v/v.

32. The method of claim 31, wherein the n-butanol is produced at a concentration of greater than 10% v/v.

33. A cell lysate produced by the method of any one of claims 1-26.

34. A method of producing n-butanol, the method comprising:
(a) culturing multiple populations of engineered cells, wherein each population of cells express enzymes of a n-butanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase;
(b) lysing populations of engineered cells cultured in step (a), thereby producing multiple cell lysates, wherein each cell lysate comprises enzymes of the n-butanol biosynthetic pathway;
(c) combining in a single reaction mixture (i) glucose, (ii) at least a portion of each of the cell lysates and, optionally, (iii) a purified enzyme of the n-butanol biosynthetic pathway, wherein the single reaction mixture includes the following enzymes of the n-butanol biosynthetic pathway: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, pyruvate dehydrogenase complex, acetyl-CoA acetyltransferase, hydroxybutyrl-CoA dehydrogenase, enoyl-CoA hydratase, crotonyl-CoA reductase, butyraldehyde dehydrogenase, and alcohol dehydrogenase; and
(d) incubating the single reaction mixture under conditions that result in production of n-butanol.

35. A method of producing a cell lysate for producing isobutanol, the method comprising:
(a) culturing engineered cells that express at least one enzyme of an isobutanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, ketol-acid reductoisomerase, dihydroxy-acid dehydratase, branched-chain-2-oxoacid decarboxylase, and alcohol dehydrogenase; and
(b) lysing engineered cells cultured in step (a), thereby producing a cell lysate that comprises at least one enzyme of the isobutanol biosynthetic pathway.

36. The method of claim 35, wherein the engineered cells express at least 2 enzymes of the isobutanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, ketol-acid reductoisomerase, dihydroxy-acid dehydratase, branched-chain-2-oxoacid decarboxylase, and alcohol dehydrogenase.

37. The method of claim 36, wherein the engineered cells express 2 to 15 enzymes of the isobutanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, ketol-acid reductoisomerase, dihydroxy-acid dehydratase, branched-chain-2-oxoacid decarboxylase, and alcohol dehydrogenase.

38. The method of claim 37, wherein the engineered cells express glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, ketol-acid reductoisomerase, dihydroxy-acid dehydratase, branched-chain-2-oxoacid decarboxylase, and alcohol dehydrogenase.

39. The method of any one of claims 35-38, wherein at least one enzyme of the isobutanol biosynthetic pathway expressed by the engineered cell is encoded by an endogenous nucleic acid.

40. The method of claim 39, wherein at least one enzyme encoded by an endogenous nucleic acid is selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase.

41. The method of any one of claims 35-40, wherein at least one enzyme of the isobutanol biosynthetic pathway expressed by the engineered cell is encoded by an engineered nucleic acid.

42. The method of claim 41, wherein at least one enzyme encoded by the engineered nucleic acid is selected from the group consisting of: acetolactate synthase, ketol-acid reductoisomerase, dihydroxy-acid dehydratase, branched-chain-2-oxoacid decarboxylase, and alcohol dehydrogenase.

43. The method of any one of claims 35-42 further comprising combining the cell lysate with at least one other cell lysate that expresses at least one enzyme of the isobutanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, ketol-acid reductoisomerase, dihydroxy-acid dehydratase, branched-chain-2-oxoacid decarboxylase, and alcohol dehydrogenase.

44. The method of any one of claims 35-43 further comprising combining the cell lysate with at least one purified enzyme of the isobutanol biosynthetic pathway.

45. The method of any one of claims 35-44 further comprising combining the cell lysate with glucose.

46. The method of claim 45 further comprising combining the cell lysate with at least one substance selected from the group consisting of: substrates, enzymes, nutrients, co-factors, buffers, and reducing agents.

47. The method of any one of claims 35-46 further comprising combining the cell lysate with a proton leakage agent.

48. The method of claim 47, wherein the proton leakage agent is dinitrophenol.

49. The method of any one of claims 35-48 further comprising combining the cell lysate with a phosphatase.

50. The method of any one of claims 35-49, wherein the engineered cells of step (a) further comprise:
an engineered nucleic acid encoding a target enzyme that negatively impacts the rate of isobutanol production and includes a site-specific protease-recognition sequence in the protein sequence of the target enzyme, and
an engineered nucleic acid encoding a site-specific protease that cleaves the site-specific protease-recognition sequence of the target enzyme and includes a periplasmic-targeting sequence, wherein the engineered cells are cultured under conditions that result in expression of enzymes and periplasmic sequestration of a site-specific protease that comprises a periplasmic-targeting sequence.

51. The method of claim 50 wherein target enzyme:
competes for substrates or cofactors with an enzyme that increases the rate of precursor supplied to the isobutanol biosynthetic pathway;
competes for substrates or cofactors with an enzyme that is a key pathway entry enzyme of the isobutanol biosynthetic pathway; or
competes for substrates or cofactors with an enzyme that supplies a substrate or cofactor of the isobutanol biosynthetic pathway.

52. The method of claim 51, wherein the target enzyme is selected from the group consisting of: pyruvate dehydrogenase, PEP carboxylase, citrate synthase, phosphate acetyltransferase, β-ketoacyl-ACP synthase III, and acetyl-CoA carboxylase.

53. The method of claim 50 or 51, wherein the engineered cells do not express an endogenous wild-type form of the target enzyme.

54. The method of any one of claims 50-53, wherein the site-specific protease is selected from the group consisting of: alanine carboxypeptidase, Armillaria mellea, astacin, bacterial leucyl aminopeptidase, cancer procoagulant, cathepsin B, clostripain, cytosol alanyl aminopeptidase, elastase, endoproteinase Brg-C, enterokinase, gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2B, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin B, venombin BB and Xaa-pro aminopeptidase.

55. The method of claim 54, wherein the site-specific protease is human rhinovirus 3C protease.

56. The method of any one of claims 50-55, wherein the nucleic acid encoding the site-specific protease is operably linked to an inducible promoter.

57. The method of any one of claims 35-56, wherein the engineered cells are engineered bacterial cells.

58. The method of claim 57, wherein the engineered bacterial cells are engineered Escherichia coli cells.

59. The method of any one of claims 35-58, wherein at least one of the enzymes of the isobutanol biosynthetic pathway is linked to a periplasmic-targeting sequence.

60. The method of any one of claims 50-59, wherein the periplasmic-targeting sequence is a sequence selected from the group consisting of:
(SEQ ID NO: 1)
MKIKTGARILALSALTTMMFSASALA;
(SEQ ID NO: 2)
MKQSTIALALLPLLFTPVTKA;
(SEQ ID NO: 3)
MMITLRKLPLAVAVAAGVMSAQAMA;
(SEQ ID NO: 4)
MNKKVLTLSAVMASMLFGAAAHA;
(SEQ ID NO: 5)
MKYLLPTAAAGLLLLAAQPAMA;
(SEQ ID NO: 6)
MKKIWLALAGLVLAFSASA;
(SEQ ID NO: 7)
MMTKIKLLMLIIFYLIISASAHA;
(SEQ ID NO: 8)
MKQALRVAFGFLILWASVLHA;
(SEQ ID NO: 9)
MRVLLFLLLSLFMLPAFS;
and
(SEQ ID NO: 10)
MANNDLFQASRRRFLAQLGGLTVAGMLGPSLLTPRRATA.

61. The method of any one of claims 45-60 further comprising incubating the cell lysate under conditions that result in production of isobutanol.

62. A method of producing isobutanol, comprising:
combining two or more of the cell lysates produced by the method of any one of claims 35-60 and, optionally, at least one purified enzyme of the isobutanol biosynthetic pathway; and
incubating the two or more cell lysates under conditions that result in production of isobutanol.

63. The method of claim 61 or 62 further comprising isolating the isobutanol.

64. The method of any one of claims 61-63, wherein the isobutanol is produced at a concentration of greater than 2% v/v.

65. The method of claim 64, wherein the isobutanol is produced at a concentration of greater than 5% v/v.

66. The method of claim 65, wherein the isobutanol is produced at a concentration of greater than 10% v/v.

67. A cell lysate produced by the method of any one of claims 35-60

68. A method of producing isobutanol, the method comprising:
(a) culturing multiple populations of engineered cells, wherein each population of cells express enzymes of an isobutanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, ketol-acid reductoisomerase, dihydroxy-acid dehydratase, branched-chain-2-oxoacid decarboxylase, and alcohol dehydrogenase;
(b) lysing populations of engineered cells cultured in step (a), thereby producing multiple cell lysates, wherein each cell lysate comprises enzymes of the isobutanol biosynthetic pathway;
(c) combining in a single reaction mixture (i) glucose, (ii) at least a portion of each of the cell lysates and, optionally, (iii) a purified enzyme of the isobutanol biosynthetic pathway, wherein the single reaction mixture includes the following enzymes of the isobutanol biosynthetic pathway: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, ketol-acid reductoisomerase, dihydroxy-acid dehydratase, branched-chain-2-oxoacid decarboxylase, and alcohol dehydrogenase; and
(d) incubating the single reaction mixture under conditions that result in production of isobutanol.

69. A method of producing a cell lysate for producing pyruvate, the method comprising:
(a) culturing engineered cells that express glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase; and
(b) lysing engineered cells cultured in step (a), thereby producing a cell lysate that comprises enzymes of the glycolytic pathway.

70. The method of claim 69, wherein at least one of the enzymes of (a) is encoded by an engineered nucleic acid.

71. The method of claim 69 or 70, wherein the engineered cells of step (a) further comprise:
an engineered nucleic acid encoding a target enzyme that competes with an enzyme in the glycolytic pathway for substrates or cofactors and includes a site-specific protease-recognition sequence in the protein sequence of the target enzyme, and
an engineered nucleic acid encoding a site-specific protease that cleaves the site-specific protease-recognition sequence of the target enzyme and includes a periplasmic-targeting sequence, wherein the engineered cells are cultured under conditions that result in expression of enzymes and periplasmic sequestration of a site-specific protease that comprises a periplasmic-targeting sequence.

72. The method of claim 71, wherein activity of the target enzyme negatively impacts the rate of pyruvate production.

73. The method of claim 72, wherein the target enzyme is selected from the group consisting of: pyruvate dehydrogenase, PEP carboxylase, citrate synthase, phosphate acetyltransferase, β-ketoacyl-ACP synthase III, and acetyl-CoA carboxylase.

74. The method of claim 72 or 73, wherein the engineered cells do not express an endogenous wild-type form of the target enzyme.

75. The method of any one of claims 71-74, wherein the site-specific protease is selected from the group consisting of: alanine carboxypeptidase, Armillaria mellea, astacin, bacterial leucyl aminopeptidase, cancer procoagulant, cathepsin B, clostripain, cytosol alanyl aminopeptidase, elastase, endoproteinase Brg-C, enterokinase, gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2B, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin B, venombin BB and Xaa-pro aminopeptidase.

76. The method of claim 75, wherein the site-specific protease is human rhinovirus 3C protease.

77. The method of any one of claims 69-76 further comprising combining the cell lysate with glucose.

78. The method of claim 77 further comprising combining the cell lysate with at least one substance selected from the group consisting of: substrates, enzymes, nutrients, co-factors, buffers, and reducing agents.

79. The method of any one of claims 69-78 further comprising combining the cell lysate with a proton leakage agent.

80. The method of claim 79, wherein the proton leakage agent is dinitrophenol.

81. The method of any one of claims 69-80 further comprising combining the cell lysate with a phosphatase.

82. A method of producing a cell lysate for producing 2-butanol, the method comprising:
(a) culturing engineered cells that express at least one enzyme of a 2-butanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, acetolactate decarboxylase, diacetyl reductase, diol dehydratase or glycerol dehydratase, and butanol dehydrogenase, wherein the cells are cultured under conditions that result in expression of enzymes; and
(b) lysing engineered cells cultured in step (a), thereby producing a cell lysate that comprises at least one enzyme of the 2-butanol biosynthetic pathway.

83. The method of claim 82, wherein the engineered cells express at least 2 enzymes of the 2-butanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, acetolactate decarboxylase, diacetyl reductase, diol dehydratase or glycerol dehydratase, and butanol dehydrogenase.

84. The method of claim 83, wherein the engineered cells express 2 to 15 enzymes of the 2-butanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, acetolactate decarboxylase, diacetyl reductase, diol dehydratase or glycerol dehydratase, and butanol dehydrogenase.

85. The method of claim 84, wherein the engineered cells express glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, acetolactate decarboxylase, diacetyl reductase, diol dehydratase or glycerol dehydratase, and butanol dehydrogenase.

86. The method of any one of claims 82-85, wherein at least one enzyme of the 2-butanol biosynthetic pathway expressed by the engineered cell is encoded by an endogenous nucleic acid.

87. The method of claim 86, wherein at least one enzyme encoded by an endogenous nucleic acid is selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, and pyruvate kinase.

88. The method of any one of claims 82-87, wherein at least one enzyme of the 2-butanol biosynthetic pathway expressed by the engineered cell is encoded by an engineered nucleic acid.

89. The method of claim 88, wherein at least one enzyme encoded by the engineered nucleic acid is selected from the group consisting of: acetolactate synthase, acetolactate decarboxylase, diacetyl reductase, diol dehydratase or glycerol dehydratase, and butanol dehydrogenase.

90. The method of any one of claims 82-89 further comprising combining the cell lysate with at least one other cell lysate that expresses at least one enzyme of the 2-butanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, acetolactate decarboxylase, diacetyl reductase, diol dehydratase or glycerol dehydratase, and butanol dehydrogenase.

91. The method of any one of claims 82-90 further comprising combining the cell lysate with at least one purified enzyme of the 2-butanol biosynthetic pathway.

92. The method of any one of claims 82-91 further comprising combining the cell lysate with glucose.

93. The method of claim 92 further comprising combining the cell lysate with at least one substance selected from the group consisting of: substrates, enzymes, nutrients, co-factors, buffers, and reducing agents.

94. The method of any one of claims 82-93 further comprising combining the cell lysate with a proton leakage agent.

95. The method of claim 94, wherein the proton leakage agent is dinitrophenol.

96. The method of any one of claims 82-95 further comprising combining the cell lysate with a phosphatase.

97. The method of any one of claims 82-96, wherein the engineered cells of step (a) further comprise:
an engineered nucleic acid encoding a target enzyme that negatively impacts the rate of 2-butanol production and includes a site-specific protease-recognition sequence in the protein sequence of the target enzyme, and
an engineered nucleic acid encoding a site-specific protease that cleaves the site-specific protease-recognition sequence of the target enzyme and includes a periplasmic-targeting sequence, wherein the engineered cells are cultured under conditions that result in expression of enzymes and periplasmic sequestration of a site-specific protease that comprises a periplasmic-targeting sequence.

98. The method of claim 97, wherein target enzyme:
competes for substrates or cofactors with an enzyme that increases the rate of precursor supplied to the 2-butanol biosynthetic pathway;
competes for substrates or cofactors with an enzyme that is a key pathway entry enzyme of the 2-butanol biosynthetic pathway; or
competes for substrates or cofactors with an enzyme that supplies a substrate or cofactor of the 2-butanol biosynthetic pathway.

99. The method of claim 98, wherein the target enzyme is selected from the group consisting of: pyruvate dehydrogenase, PEP carboxylase, citrate synthase, phosphate acetyltransferase, β-ketoacyl-ACP synthase III, and acetyl-CoA carboxylase.

100. The method of claim 98 or 99, wherein the engineered cells do not express an endogenous wild-type form of the target enzyme.

101. The method of any one of claims 97-100, wherein the site-specific protease is selected from the group consisting of: alanine carboxypeptidase, Armillaria mellea, astacin, bacterial leucyl aminopeptidase, cancer procoagulant, cathepsin B, clostripain, cytosol alanyl aminopeptidase, elastase, endoproteinase Brg-C, enterokinase, gastricsin, gelatinase, Gly-X carboxypeptidase, glycyl endopeptidase, human rhinovirus 3C protease, hypodermin C, Iga-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2B, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin B, venombin BB and Xaa-pro aminopeptidase.

102. The method of claim 101, wherein the site-specific protease is human rhinovirus 3C protease.

103. The method of any one of claims 97-102, wherein the nucleic acid encoding the site-specific protease is operably linked to an inducible promoter.

104. The method of any one of claims 82-103, wherein the engineered cells are engineered bacterial cells.

105. The method of claim 104, wherein the engineered bacterial cells are engineered Escherichia coli cells.

106. The method of any one of claims 82-105, wherein at least one of the enzymes of the 2-butanol biosynthetic pathway is linked to a periplasmic-targeting sequence.

107. The method of any one of claims 97-106, wherein the periplasmic-targeting sequence is a sequence selected from the group consisting of:
(SEQ ID NO: 1)
MKIKTGARILALSALTTMMFSASALA;
(SEQ ID NO: 2)
MKQSTIALALLPLLFTPVTKA;
(SEQ ID NO: 3)
MMITLRKLPLAVAVAAGVMSAQAMA;
(SEQ ID NO: 4)
MNKKVLTLSAVMASMLFGAAAHA;
(SEQ ID NO: 5)
MKYLLPTAAAGLLLLAAQPAMA;
(SEQ ID NO: 6)
MKKIWLALAGLVLAFSASA;
(SEQ ID NO: 7)
MMTKIKLLMLIIFYLIISASAHA;
(SEQ ID NO: 8)
MKQALRVAFGFLILWASVLHA;
(SEQ ID NO: 9)
MRVLLFLLLSLFMLPAFS;
and
(SEQ ID NO: 10)
MANNDLFQASRRRFLAQLGGLTVAGMLGPSLLTPRRATA.

108. The method of any one of claims 92-107 further comprising incubating the cell lysate under conditions that result in production of 2-butanol.

109. A method of producing 2-butanol, comprising:
combining two or more of the cell lysates produced by the method of any one of claims 1-79-103 and, optionally, at least one purified enzyme of the 2-butanol biosynthetic pathway; and
incubating the two or more cell lysates under conditions that result in production of 2-butanol.

110. The method of claim 108 or 109 further comprising isolating the 2-butanol.

111. The method of any one of claims 108-110, wherein the 2-butanol is produced at a concentration of greater than 2% v/v.

112. The method of claim 111, wherein the 2-butanol is produced at a concentration of greater than 5% v/v.

113. The method of claim 112, wherein the 2-butanol is produced at a concentration of greater than 10% v/v.

114. A cell lysate produced by the method of any one of claims 82-107.

115. A method of producing 2-butanol, the method comprising:
(a) culturing multiple populations of engineered cells, wherein each population of cells express enzymes of a 2-butanol biosynthetic pathway selected from the group consisting of: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, acetolactate decarboxylase, diacetyl reductase, diol dehydratase or glycerol dehydratase, and butanol dehydrogenase;
(b) lysing populations of engineered cells cultured in step (a), thereby producing multiple cell lysates, wherein each cell lysate comprises enzymes of the 2-butanol biosynthetic pathway;
(c) combining in a single reaction mixture (i) glucose, (ii) at least a portion of each of the cell lysates and, optionally, (iii) a purified enzyme of the 2-butanol biosynthetic pathway, wherein the single reaction mixture includes the following enzymes of the 2-butanol biosynthetic pathway: glucokinase, phosphoglucose isomerase, phosphofructokinase, fructose bisphosphate aldolase, triose phosphate isomerase, glyceraldehyde 3-phosphate dehydrogenase, phosphoglycerate kinase, phosphoglycerate mutase, enolase, pyruvate kinase, acetolactate synthase, acetolactate decarboxylase, diacetyl reductase, diol dehydratase or glycerol dehydratase, and butanol dehydrogenase; and
(d) incubating the single reaction mixture under conditions that result in production of 2-butanol.
